(19) United States
(12) Patent Application Publication
(10) Pub. No.: US 2025/0367662 A1
(43) Pub. Date: Dec. 4, 2025

(54) SYSTEMS AND METHODS FOR PERFORMING BIOLOGICAL ASSAYS USING A THERMALLY SEALED VALVE

(71) Applicant: PFIZER INC., New York, NY (US)

(72) Inventors: David Alexander Rolfe, San Francisco, CA (US); Aman Adam Ullah, Moraga, CA (US); Max Kevin McGee, San Francisco, CA (US); Anne Preut, Oakland, CA (US); Gautam Batra, Cupertino, CA (US); James Gregory Provins, Richmond, CA (US); Debkishore Mitra, Fremont, CA (US)

(21) Appl. No.: 18/874,325

(22) PCT Filed: Jun. 12, 2023
(86) PCT No.: PCT/US2023/068321

§ 371 Date: Dec. 12, 2024

Related U.S. Application Data
(60) Provisional application No. 63/351,427, filed on Jun. 12, 2022.

(51) Int. Cl. B01L 3/00; C12Q 1/6806; C12Q 1/6844

(52) U.S. Cl. CPC B01L 3/502738; C12Q 1/6806; C12Q 1/6844; B01L 2200/0684; B01L 2200/0689; B01L 2200/143; B01L 2200/16; B01L 2300/042; B01L 2300/0672; B01L 2300/1805; B01L 2400/0677

(57) ABSTRACT

Provided herein are systems and methods for performing biological assays using a thermally sealed valve and/or incubation chamber. The systems and methods determine one or more characteristics of a nucleic acid amplification sample based on a modified optical property of the sample.

### CROSS-REFERENCE TO RELATED APPLICATIONS

This application claims priority to and the benefit of U.S. Provisional Patent Application No. 63/351,427, filed on Jun. 12, 2022, the entire contents of which are incorporated by reference herein for all purposes.

### INTRODUCTION

Biological sample assays are used to evaluate one or more characteristics of biological samples. Such assays can qualitatively assess and/or quantitatively measure the presence, amount and/or functional activity of one or more analytes in a biological sample. Such an assessment can be made based on a change or lack of a change occurring in the assay. For example, a change in color and/or transmittance of a biological sample or aspect thereof occurring under specific conditions during an assay can serve as an indicator of one or more characteristics of the assayed sample.

### SUMMARY

Systems and methods for performing biological assays on cells that require lysing are provided herein. The systems and methods herein utilize a thermally sealed valve to isolate and fluidically connect an incubation chamber and downstream reaction chambers. The systems and methods determine one or more characteristics of a nucleic acid amplification sample based on a modified optical property of the sample.

In one aspect, the present disclosure provides a system for performing a biological assay, the system comprising one or more of:
a. a thermal mixing module comprising:
   i. a sample receiving module for receiving a sample solution comprising a liquid sample (e.g., biological sample) and a preparation solution; and
   ii. an incubation chamber in fluidic communication with the sample receiving module;
b. a wax valve channel in fluidic communication with the incubation chamber;
c. an optical property modifying (OPM) module operatively coupled with the incubation chamber via the wax valve channel, the OPM module comprising one or more reaction chambers each comprising an assay reagent;
d. a thermally sealed valve disposed i) within the wax valve channel, and ii) between the sample receiving module and the OPM module;
e. a mixing heater configured to supply heat to the incubation chamber.
f. a valve heater; and
g. a reaction heater.

In some embodiments, the incubation chamber comprises a reagent or a lytic agent. As described herein, a reagent is also contemplated wherever a lytic agent is contemplated. In some embodiments, the mixing heater is configured to be aligned with at least a portion of the thermal mixing module and offset from a center portion of the incubation chamber.

In some embodiments, the system further comprises a sample preparation device configured to mate with the sample receiving module. In some embodiments, the sample receiving module comprises a puncturing element configured to pierce a breakable seal on the sample preparation device, thereby enabling fluidic communication between a sample preparation chamber within the sample preparation device and the incubation chamber.

In some embodiments, the breakable seal comprises a foil. In some embodiments, the sample receiving module comprises a luer for coupling the sample preparation tube to the assay device. In some embodiments, the sample preparation tube comprises a collar that contacts the luer of the sample receiving module, so as to form a leak-tight seal between the sample preparation device and the sample receiving module when mated together. In some embodiments, the incubation chamber comprises a vent. In some embodiments, the vent is a selective venting element. In some embodiments, the selective venting element is a rigid or semi-rigid porous matrix comprising (e.g., embedded therein is) a material that swells upon contact with liquid. In some embodiments, the selective venting element is a self-sealing polymer comprising (e.g., embedded therein is) a material that swells upon contact with liquid. In some embodiments, the selective venting element is a self-sealing sintered polymer vent plug. In some embodiments, the selective venting element is a self-sealing fibrous material comprising (e.g., embedded therein is) a material that swells upon contact with liquid. In some embodiments, the selective venting element is a self-sealing porous polyethylene vent comprising an embedded hydrogel. In some embodiments, the selective venting element is a thermoplastic (e.g., temperature resistant). In some embodiments, the selective venting element is polytetrafluoroethylene or polyethersulfone. In some embodiments, the selective venting element comprises heat staked material(s). In some embodiments, the selective venting element comprises a hydrophobic porous membrane. In some embodiments, the vent comprises a sensing channel in fluidic communication with a fill-detection chamber configured to detect liquid filling thereof.

In some embodiments, the thermal mixing module further comprises one or more sensors configured to i) detect an initial presence of liquid within the incubation chamber, ii) detect a liquid level in the incubation chamber, iii) or both. In some embodiments, the one or more sensors comprises a capacitive sensor disposed below the incubation chamber. In some embodiments, the one or more sensors comprises one or more bottom electrodes in operative communication with a circuit board and configured to penetrate through a bottom wall of the incubation chamber so as to be exposed to within the incubation chamber.

In some embodiments, the thermal mixing module comprises a light source, optionally a light emitting diode (LED) or a laser.

In some embodiments, the system further comprises one or more top electrodes in operative communication with the circuit board and configured to penetrate through a wall other than the bottom wall of the incubation chamber, so as to be exposed to within the incubation chamber and thereby detect a liquid level within the incubation chamber. In some embodiments, the thermal mixing module comprises an electrode socket comprising an electrode (e.g., top electrode; see for example FIG. 5). In some embodiments, a sensor of the one or more sensors is disposed within a fill-detection chamber or otherwise operatively connected to a fill-detection chamber (e.g., the sensor can be positioned within the sample receiving module but not within the fill detection chamber, however, the sensor can still detect light transmitted from and/or through the fill-detection chamber). In some embodiments, the fill-detection chamber is in fluidic communication with the incubation chamber via a sensing channel, and one or more sensors is configured to detect a change in light within the fill-detection chamber. In some embodiments, the one or more sensors comprises a thermocouple coupled to the mixing heater and/or a portion of the incubation chamber, so as to detect a change in a temperature of the mixing heater and/or incubation chamber, thereby correlating with a presence of a liquid within the incubation chamber. In some embodiments, the one or more sensors are in operative communication with the mixing heater. In some embodiments, the one or more sensors function as an interlock for the mixing heater, such that the mixing heater is configured to be activated and/or deactivated based on detection of a liquid and/or a liquid level within the incubation chamber by the one or more sensors.

In some embodiments, the incubation chamber comprises a light source (e.g., a light emitting diode (LED), a laser, etc.). As used herein, a “light source” refers to an element that is capable of individually illuminating a surface.

In some embodiments, the lytic agent comprises a lyophilized pellet. In some embodiments, the lytic agent comprises Dithiothreitol (DTT), Proteinase K, Mutanolysin, Lysostaphin, Lysozyme, or a combination thereof. In some embodiments, the lytic agent comprises one or more surfactants. In some embodiments, the one or more surfactants comprises a polysorbate. In some embodiments, lytic agent comprises one or more components of a buffer solution. In some embodiments, the mixing heater is configured to heat the sample solution within the incubation chamber, thereby enabling mixing of the sample solution and the lytic agent therein to form a prepared sample solution.

In some embodiments, the incubation chamber has one or more rounded edges. In some embodiments, the one or more rounded edges enables the sample solution to circulate or substantially circulate within the incubation chamber when receiving heat from the mixing heater. In some embodiments, a height of the incubation chamber relative to a width of the incubation chamber is prescribed to minimize and/or reduce areas of dead volume within the incubation chamber. In some embodiments, a length of the incubation chamber is from about 0.5 times to about 3 times the height of the incubation chamber. In some embodiments, a length of the incubation chamber is about 0.4 times, 0.5 times, 0.6 times, 0.7 times, 0.8 times, 0.9 times, 1 times, 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, or 3.5 times the height of the incubation chamber. In some embodiments, the width of the incubation chamber is from about ⅛ times to about 1.0 times an average of the length and the width of the incubation chamber. In some embodiments, the width of the incubation chamber is from about ⅛ times, 1/7 times, ⅙ times, ⅕ times, ¼ times, ⅓ times, ½ times to about 1.0 times an average of the length and the width of the incubation chamber. In some embodiments, the incubation chamber has a volume from about 0.1 mL to about 10 mL, such as from about 0.5 mL to about 5 mL. In some embodiments, the incubation chamber has a volume from about 0.1 mL to 0.2 mL, 0.1 mL to 0.3 mL, 0.1 mL to 0.4 mL, 0.1 mL to 0.5 mL, 0.1 mL to 0.6 mL, 0.1 mL to 0.7 mL, 0.1 mL to 0.8 mL, or 0.1 mL to 0.9 mL. In some embodiments, the incubation chamber has a volume from about 0.1 mL to 1 mL, 0.1 mL to 2 mL, 0.1 mL to 3 mL, 0.1 mL to 4 mL, 0.1 mL to 5 mL, 0.1 mL to 6 mL, 0.1 mL to 7 mL, 0.1 mL to 8 mL, 0.1 mL to 9 mL, 0.1 mL to 10 mL. In some embodiments, the incubation chamber has a volume from about 0.5 mL to 1 mL, 0.5 mL to 2 mL, 0.5 mL to 3 mL, 0.5 mL to 4 mL, 0.5 mL to 5 mL, 0.5 mL to 6 mL, 0.5 mL to 7 mL, 0.5 mL to 8 mL, 0.5 mL to 9 mL, or 0.5 mL to 10 mL. In some embodiments, the incubation chamber has a volume of about 0.1 mL, 0.2 mL, 0.3 mL, 0.4 mL, 0.5 mL, 0.6 mL, 0.7 mL, 0.8 mL, 0.9mL, 1 mL, 1.1 mL, 1.2 mL, 1.3 mL, 1.4 mL, 1.5 mL, 1.6 mL, 1.7 mL, 1.8 mL, 1.9 mL, 2 mL, 2.1 mL, 2.2 mL, 2.3 mL, 2.4 mL, 2.5 mL, 2.6 mL, 2.7 mL, 2.8 mL, 2.9 mL, 3 mL, 3.1 mL, 3.2 mL, 3.3 mL, 3.4 mL, 3.5 mL, 3.6 mL, 3.7 mL, 3.8 mL, 3.9 mL, 4 mL, 4.1 mL, 4.2 mL, 4.3 mL, 4.4 mL, 4.5 mL, 4.6 mL, 4.7 mL, 4.8 mL, 4.9 mL, 5 mL, 5.1 mL, 5.2 mL, 5.3 mL, 5.4 mL, 5.5 mL, 5.6 mL, 5.7 mL, 5.8 mL, 5.9 mL, 6 mL, 6.1 mL, 6.2 mL, 6.3 mL, 6.4 mL, 6.5 mL, 6.6 mL, 6.7 mL, 6.8 mL, 6.9 mL, 7 mL, 7.1 mL, 7.2 mL, 7.3 mL, 7.4 mL, 7.5 mL, 7.6 mL, 7.7 mL, 7.8 mL, 7.9 mL, 8 mL, 8.1 mL, 8.2 mL, 8.3 mL, 8.4 mL, 8.5 mL, 8.6 mL, 8.7 mL, 8.8 mL, 8.9 mL, 9 mL, 9.1 mL, 9.2 mL, 9.3 mL, 9.4 mL, 9.5 mL, 9.6 mL, 9.7 mL, 9.8 mL, 9.9 mL, or 10 mL.

In some embodiments, the mixing heater is configured to heat the sample solution within the incubation chamber for a prescribed amount of time. In some embodiments, the prescribed amount of time is about 5 minutes. In some embodiments, the prescribed amount of time is from about 30 seconds to about 20 minutes. In some embodiments, the prescribed amount of time is from about 1 minute to about 10 minutes In some embodiments, the prescribed amount of time is from about 1 minute to 5 minutes or from about 5 minutes to 10 minutes. In some embodiments, the prescribed amount of time is about 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, or 59 seconds. In some embodiments, the prescribed amount of time is about 1 minute, 2 minutes, 3 minutes, 4 minutes, 5 minutes, 6 minutes, 7 minutes, 8 minutes, 9 minutes, or 10 minutes. In some embodiments, the prescribed amount of time is about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, or 25 minutes. In some embodiments, the prescribed amount of time is from about 1 minute to 2 minutes, 1 minute to 3 minutes, 1 minute to 4 minutes, 1 minute to 5 minutes, 1 minute to 6 minutes, 1 minute to 7 minutes, 1 minute to 8 minutes, 1 minute to 9 minutes, 1 minute to 10 minutes, 1 minute to 11 minutes, 1 minute to 12 minutes, 1 minute to 13 minutes, 1 minute to 14 minutes, 1 minute to 15 minutes, 1 minute to 16 minutes, 1 minute to 17 minutes, 1 minute to 18 minutes, 1 minute to 19 minutes, or 1 minute to 20 minutes. In some embodiments, the prescribed amount of time is from about 5 minute to 6 minutes, 5 minutes to 7 minutes, 5 minutes to 8 minutes, 5 minutes to 9 minutes, 5 minutes to 10 minutes, 5 minutes to 11 minutes, 5 minutes to 12 minutes, 5 minutes to 13 minutes, 5 minutes to 14 minutes, 5 minutes to 15 minutes, 5 minutes to 16 minutes, 5 minutes to 17 minutes, 5 minutes to 18 minutes, 5 minutes to 19 minutes, or 5 minutes to 20 minutes. In some embodiments, the prescribed amount of time is from about 10 minute to 11 minutes, 10 minutes to 12 minutes, 10 minutes to 13 minutes, 10 minutes to 14 minutes, 10 minutes to 15 minutes, 10 minutes to 16 minutes, 10 minutes to 17 minutes, 10 minutes to 18 minutes, 10 minutes to 19 minutes, or 10 minutes to 20 minutes.

In some embodiments, the wax valve channel is located at an end of the incubation chamber opposite to the sample receiving module. In some embodiments, the thermally sealed valve comprises a wax. In some embodiments, the wax is water-soluble. In some embodiments, the wax comprises a water-soluble polymer. In some embodiments, the thermally sealed valve comprises a polymer. In some embodiments, the polymer is water soluble. In some embodiments, the polymer comprises polyethylene glycol (PEG). In some embodiments, the thermally sealed valve has a molecular weight from about 1,300 g/mol to about 10,000 g/mol. In some embodiments, the thermally sealed valve has a molecular weight of about 6,000 g/mol.

In some embodiments, the thermally sealed valve has a melting temperature of about 40° C. to about 75° C. In some embodiments, the thermally sealed valve has a melting temperature of about 40° C. to about 45° C., about 40° C. to about 50° C., about 40° C. to about 55° C., about 40° C. to about 60° C., about 40° C. to about 65° C., about 40° C. to about 70° C., about 40° C. to about 75° C., or about 40° C. to about 80° C. In some embodiments, the thermally sealed valve has a melting temperature of about 35° C. to about 40° C., about 35° C. to about 45° C., about 35° C. to about 50° C., about 35° C. to about 55° C., about 35° C. to about 60° C., about 35° C. to about 65° C., about 35° C. to about 70° C., about 35° C. to about 75° C., or about 35° C. to about 80° C. In some embodiments, the thermally sealed valve has a melting temperature of about 35° C., 36° C., 37°° C., 38° C., 39° C., 40° C., 41° C., 42° C., 43° C., 44° C., or 45° C. In some embodiments, the thermally sealed valve has a melting temperature of about 35° C., 40° C., 45° C., 50° C., 55° C., 60° C., 65° C., 70° C., 75° C., or 80° C.

In some embodiments, the thermally sealed valve has a volume of from about 2 uL to about 6 uL within the wax valve channel. In some embodiments, the thermally sealed valve has a volume of from about 3 uL to about 5 uL within the wax valve channel. In some embodiments, the thermally sealed valve has a volume of from about 3 uL to about 4 uL within the wax valve channel. In some embodiments, the thermally sealed valve has a volume of from about 2 uL to about 5 uL within the wax valve channel. In some embodiments, the thermally sealed valve has a volume of from about 2 uL to about 4 uL within the wax valve channel. In some embodiments, the thermally sealed valve has a volume of from about 2 uL to about 3 uL within the wax valve channel. In some embodiments, the thermally sealed valve has a volume of from about 2 uL within the wax valve channel. In some embodiments, the thermally sealed valve has a volume of from about 3 uL within the wax valve channel. In some embodiments, the thermally sealed valve has a volume of from about 4 uL within the wax valve channel. In some embodiments, the thermally sealed valve has a volume of from about 5 uL within the wax valve channel. In some embodiments, the thermally sealed valve has a volume of from about 6 uL within the wax valve channel. In some embodiments, the thermally sealed valve has a volume of from about 7 uL within the wax valve channel. In some embodiments, the wax valve channel comprises a valve fill port for receiving the valve therein. In some embodiments, the thermally sealed valve is solid or substantially solid at a first temperature, so as to help prevent the sample solution from flowing through the wax valve channel. In some embodiments, the thermally sealed valve is configured to transition from the solid or substantially solid configuration to a soft, dissolved, and/or melted configuration after receiving sufficient heat.

In some embodiments, the system further comprises a valve heater configured to heat the thermally sealed valve, thereby enabling the thermally sealed valved to be softened, dissolved, and/or melted to allow the sample solution to flow therethrough. In some embodiments, an entrance to the wax valve channel from the incubation chamber comprises one or more converging walls. In some embodiments, the system further comprises one or more thermal conductive pads is operatively coupled with the mixing heater, the valve heater, or both. In some embodiments, the one or more thermal conductive pads (e.g., thermal gap pad) comprise a valve thermal conductive pad configured to transfer heat from the valve heater to the wax valve channel, so as to heat the thermally sealed valve. The thermal conductive pad (e.g., thermal gap pads) allows for variations in the distance between the substrate and the thermally sealed valve or incubation chamber by filling the space and conducting heat).

In some embodiments, a liquid level of the sample solution within the incubation chamber is located a higher elevation than the wax valve channel, such that the thermally sealed valve is under hydrostatic pressure from the sample solution. In some embodiments, the sample preparation tube is positioned at a higher elevation than the thermally sealed valve or the wax valve channel, such that the thermally sealed valve is under hydrostatic pressure from the sample solution. In some embodiments, the thermally sealed valve is configured to dissolve into the sample solution. In some embodiments, the prepared sample solution is configured to enter at least one reaction chamber of the one or more reaction chambers after passing through the wax valve channel. In some embodiments, the dissolved thermally sealed valve enters with the prepared sample solution in at least one reaction chamber.

In some embodiments, the system further comprises a sequestration chamber located downstream the wax valve channel and upstream the one or more reaction chambers, wherein the sequestration chamber is configured to receive the initial flow of the prepared sample solution and dissolved thermally sealed valve therein, so as to reduce the amount of the dissolved thermally sealed valve found in the one or more reaction chambers. In some embodiments, the sequestration chamber comprises a vent. In some embodiments, the chamber has an outlet and is indirectly connected to a vent.

In some embodiments, the system further comprises one or more mixing chambers (e.g., shuttle mixing chambers) so as to improve distribution of the dissolved thermally sealed valve across the one or more reaction chambers.

In some embodiments, the system further comprises a substrate operatively coupled to the thermal mixing module, the wax valve channel, and/or the OPM module. In some embodiments, the substrate comprises a printed circuit board. In some embodiments, the mixing heater and/or the valve heater is disposed on the substrate. In some embodiments, the substrate further comprises a power source operatively connected to the mixing heater and/or the valve heater. In some embodiments, the substrate includes a controller to regulate power supplied to the mixing heater and/or the valve heater so as to maintain the mixing heater and/or the valve heater substantially at a predetermined temperature. In some embodiments, the power source is configured to supply power to the mixing heater and/or the valve heater at a substantially constant rate. In some embodiments, the substrate comprises a thermal gap pad.

In some embodiments, the preparation solution is a nucleic acid amplification preparation solution. In some embodiments, the preparation solution further comprises an optical property modifying reagent. In some embodiments, the liquid sample (e.g., biological sample) comprises human saliva, urine, human mucus, vaginal fluid, seminal fluid, blood, an oral rinse or a solid tissue such as buccal tissue, bacteria, one or more spores, one or more viruses, or a combination thereof, and/or a concentrate thereof.

In some embodiments, the OPM module comprises a reaction chamber channel in fluidic communication with the wax valve channel, wherein the one or more reaction chambers are in fluidic communication with the reaction chamber channel through a corresponding branch. In some embodiments, each reaction chamber is substantially equidistant from a single sensing region disposed in the OPM module. In some embodiments, the OPM module further comprises a first plurality of light pipes, each first light pipe capable of transmitting light between one of the one or more reaction chambers and the single sensing region. In some embodiments, the OPM module further comprises a reaction heater configured to heat the one or more reaction chambers.

In some embodiments, the assay reagent comprises dried or lyophilized reagents. In some embodiments, the assay reagent comprises a nucleic acid amplification enzyme and a DNA primer.

In another aspect, the present disclosure provides a method for determining one or more characteristics of a nucleic acid amplification sample based on a modified optical property of a liquid sample (e.g., biological sample), the method comprising:
a. providing the liquid sample (e.g., biological sample) comprising a nucleic acid;
b. combining the liquid sample (e.g., biological sample) with a preparation solution comprising a buffer solution and/or an optical property modifying reagent solution, so as to produce a sample solution;
c. dispensing the sample solution into an incubation chamber;
d. mixing the sample solution with a lytic agent using a mixing heater to apply heat to the incubation chamber, so as to enable thermal mixing, thereby forming a prepared sample solution;
e. heating a thermally sealed valve disposed within a wax valve channel in fluidic communication with the incubation chamber, so as enable the prepared sample solution to flow through the wax valve channel to one or more reaction chambers comprising an assay reagent, wherein the prepared sample solution is mixed with the assay reagent to form a reaction mixture;
f. heating the reaction mixture to promote a nucleic acid amplification reaction using the nucleic acid present in the liquid sample (e.g., biological sample) and the assay reagents, the reaction generating an amplified nucleic acid and a plurality of protons;
g. reacting the protons with the optical property modifying reagent, wherein the reacting is capable of modifying an optical property of the optical property modifying reagent to allow detection of the modified optical property, which is indicative of a presence of a suspected analyte in the liquid sample (e.g., biological sample); and
h. causing a plurality of light emitting elements to emit light in a repeating pattern at a repetition frequency, so as to determine one or more characteristics of the liquid sample (e.g., biological sample) using a photosensor based on the modified optical property.

In some embodiments, the method further comprises displaying the determined characteristics using an electronic result display mechanism.

In some embodiments, providing the liquid sample (e.g., biological sample) comprises performing a nasal swab on a subject, performing a tonsil and/or throat swab on the subject, performing a vaginal swab on the subject, obtaining a hair sample from the subject, obtaining a blood draw from the subject, obtaining a urine sample from the subject, or a combination thereof.

In some embodiments, combining the liquid sample (e.g., biological sample) and the preparation solution is within a sample preparation device.

In some embodiments, the method further comprises providing a system as described in the present disclosure.

In some embodiments, dispensing the sample solution into the incubation chamber comprises coupling the sample preparation device with the sample receiving module, so as to create a fluidic pathway between the sample preparation device and the incubation chamber.

In some embodiments, the method further comprises breaking and/or rupturing a breakable seal on the sample preparation device, so as to enable the sample solution to flow from the sample preparation device to the incubation chamber.

In some embodiments, the method further comprising maintaining the mixing heater in a deactivated state until the sample solution is detected within the incubation chamber and/or until a minimum liquid level of the sample solution within the incubation chamber is detected.

In some embodiments, the sample solution is detected within the incubation chamber and/or until a minimum liquid level of the sample solution in the incubation chamber is detected using a sensor. In some embodiments, the sample solution is mixed with the lytic agent for a prescribed amount of time. In some embodiments, the prescribed amount of time is from about 1 minute to about 20 minutes.

In some embodiments, the method further comprises regulating the mixing heater based on i) a constant or substantially constant power supplied to the mixing heater, via a power supply, or ii) maintaining a constant or substantially constant temperature of the mixing heater or a portion of the incubation chamber. In some embodiments, heating the thermally sealed valve comprises activating the valve heater after the prescribed amount of time. In some embodiments, heating the thermally sealed valve results in softening, melting, and/or dissolving the thermally sealed valve, wherein said dissolving is within the prepared sample solution. In some embodiments, the thermally sealed valve is any valve described herein.

In some embodiments, the method further comprises sequestering an initial amount of volume of the prepared sample solution and the dissolved thermally sealed valve in a sequestration chamber located upstream the one or more reaction chambers.

Another aspect of the present disclosure provides a method for preparing a thermally sealed valve a system, the method comprising:
a. dispensing a melted or dissolved wax into the wax valve channel through a valve fill port;
b. sealing the valve fill port; and
c. drying or cooling the wax valve channel so as to solidify or substantially solidify the wax.

In some embodiments, the wax is dispensed into the wax valve channel using a wax dispenser. In some embodiments, sealing the valve fill port comprising using a polymer, an obstruction, a stopper, heat staking of the port, and/or a pressure-sensitive adhesive.

Another aspect of the present disclosure provides a kit for performing a biological assay, the kit comprising:
a. a sample preparation device; and
b. an assay device comprising:
   i. a thermal mixing module comprising:
      a sample receiving module for receiving a sample solution comprising a liquid sample (e.g., biological sample) and a preparation solution; and
      an incubation chamber in fluidic communication with the sample receiving module and comprising a lytic agent;
   ii. a wax valve channel in fluidic communication with the incubation chamber;
   iii. an optical property modifying (OPM) module operatively coupled with the incubation chamber via the wax valve channel, the OPM module comprising one or more reaction chambers each comprising an assay reagent;
   iv. a thermally sealed valve disposed i) within the wax valve channel, and ii) between the sample receiving module and the OPM module; and
   v. a mixing heater configured to supply heat to the incubation chamber.

In some embodiments, the assay device further comprises a valve heater. In some embodiments, the assay device further comprises a reaction heater.

In some embodiments, the sample preparation device comprises a sample collector configured to obtain a liquid sample (e.g., biological sample) and a tube comprising a preparation solution therein, and configured to receive at least a portion of the sample collector.

In some embodiments, the sample collector comprises a nasal swab, a throat and/or tonsil swab, a vaginal swab, or a combination thereof. In some embodiments, the assay device comprises any feature described in the systems or methods of the present disclosure.

In one aspect, the present disclosure provides a kit comprising an assay device and/or sample preparation device or component thereof, as disclosed herein. In some embodiments, the kit comprises instructions for use or a QR code or bar code providing a user access to instructions for use.

### BRIEF DESCRIPTION OF THE SEVERAL VIEWS OF THE DRAWINGS

FIGS. 1A and 1B provide a perspective view of an exemplary system for performing a biological assay, having a sample preparation device and assay device, according to an embodiment described herein. FIG. 1C provides a side view of an exemplary assay device, having a thermal mixing module, optical property modifying (OPM) module, and printed circuit board (PCB), according to an embodiment described herein. FIG. 1D provides a perspective view of an exemplary system for performing a biological assay, having a sample preparation device and assay device, according to an embodiment described herein. FIG. 1E provides a side view of an exemplary assay device, having a thermal mixing module, optical property modifying (OPM) module, and printed circuit board (PCB), according to an embodiment described herein.

FIG. 2A provides a top view of a bottom portion of an assay device, according to an embodiment described herein. FIG. 2B provides a top view of a bottom portion of an assay device, according to an alternative embodiment described herein.

FIG. 3A provides a side cutaway view of an image depicting a sample preparation device coupled with an assay device, according to an embodiment described herein. FIG. 3B provides a side cutaway view of an image depicting a sample preparation device coupled with an assay device, according to an alternative embodiment described herein

FIG. 4 provides an exemplary side cutaway view of an image depicting a coupled sample receiving module and sample preparation device, according to an embodiment described herein.

FIG. 5 provides another exemplary side cutaway view of an image depicting a sample preparation device coupled with an assay device, showing top and bottom electrodes, according to an embodiment described herein.

FIG. 6 provides an exemplary side cutaway view of an image depicting the assay device, showing incubation chamber, wax valve channel, and thermal pad for the valve heater, according to an embodiment described herein.

FIG. 7 provides a graph of an incubation chamber heater and wall temperature profile based on various fill conditions.

FIG. 8 provides an exemplary front view of an image for a wax dispenser device, according to an embodiment described herein.

FIG. 9 provides an exemplary process flow for filling a wax valve channel with a wax, according to an embodiment described herein.

FIG. 10 provides an exemplary process flow for addition of fluid to the incubation chamber and dissolution of the thermally sealed valve material after application of heat, according to an embodiment described herein.

FIG. 11 provides an exemplary flow chart of a method for performing a biological assay using a system described herein, according to an embodiment described herein.

FIG. 12 provides a graphical representation of the concentration of a dissolved valve material detected in the plurality of reaction chambers, according to an embodiment described herein.

FIG. 13 provides a graphical representation of dissolved valve material detected in the plurality of reaction chambers with or without a sequestration chamber.

FIG. 14 provides a top view of testing apparatus for evaluating actuation temperature and movement of thermally sealed valve material in a channel.

FIG. 15 provides a graphical representation of the actuation temperature for thermally sealed valve material (e.g., PEG) at differing environmental conditions.

FIGS. 16 and 17 each provides a top view of a sample receiving module, showing two different wax valve channel geometries, according to embodiments described herein.

FIG. 18 provides a top view of an exemplary fill detection chamber having a light source and photosensor to detect liquid entry/chamber filling.

FIG. 19 provides a top view of an aluminum cylinder secured by three crush ribs.

### DETAILED DESCRIPTION

Systems and methods for performing biological assays are provided herein. The systems and methods determine one or more characteristics of a nucleic acid amplification sample based on a modified optical property of the eluted sample. The systems and methods herein utilize a thermally sealed valve comprising wax to isolate the contents of an incubation chamber and downstream reaction chamber(s). The wax is dissolved to allow fluidic connection between the incubation chamber and downstream reaction chamber(s).

Before the present invention(s) are described in greater detail, it is to be understood that this invention(s) is not limited to particular embodiments described, as such can, of course, vary. It is also to be understood that the terminology used herein is for the purpose of describing particular embodiments only, and is not intended to be limiting, since the scope of the present invention(s) will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges can independently be included in the smaller ranges and are also encompassed within the present disclosure, subject to any specifically excluded limit in the stated range. Where the stated range includes one or both of the limits, ranges excluding either or both of those included limits are also included in the present disclosure.

Certain ranges can be presented herein with numerical values being preceded by the term “about.” As used in this specification and the claims, unless otherwise stated, the term “about,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, or +/−15%, depending on the embodiment. As a non-limiting example, about 100 meters represents a range of 95 meters to 105 meters, 90 meters to 110 meters, or 85 meters to 115 meters depending on the embodiments.

The term “substantially” refers to less than or equal to +/−1%, +/−2%, +/−3%, +/−4%, +/−5%, +/−6%, +/−7%, +/−8%, +/−9%, +/−10%, +/−11%, +/−12%, +/−14%, or +/−15% variation. As a non-limiting example, substantially parallel represents a range of −1 to 1 degree difference, −5 to 5 degree difference, or −15 degrees to 15 degrees of difference from being parallel, depending on the embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided can be different from the actual publication dates which can need to be independently confirmed.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

In further describing the subject invention, subject devices for use in practicing the subject systems will be discussed in greater detail, followed by a review of associated methods.

### Definitions

Terms used in the claims and specification are defined as set forth below unless otherwise specified. Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “colorimetry” or “colorimetric” refers to techniques of quantifying or otherwise observing colored compound concentrations in solution. “Colorimetric detection” refers to any method of detecting such colored compounds and/or the change in color of the compounds in solution. Methods can include visual observation, absorbance measurements, or fluorescence measurements, among others.

The term “halochromic agent” refers to a composition that changes color upon some chemical reaction. In particular, a halochromic agent can refer to a composition that changes color with a pH change. Different halochromic agents can change colors over different pH transition ranges.

The term “transition pH range” or “pH transition range” refers to a pH range over which the color of a particular sample or compound changes. A specific transition pH range for a sample can depend on a halochromic agent in the sample (see above).

The term “nucleic acid amplification” or “amplification reaction” refers to methods of amplifying DNA, RNA, or modified versions thereof. Nucleic acid amplification includes several techniques, such as an isothermal reaction or a thermocycled reaction. More specifically, nucleic acid amplification includes methods such as polymerase chain reaction (PCR), loop-mediated isothermal amplification (LAMP), strand displacement amplification (SDA), recombinase polymerase amplification (RPA), helicase dependent amplification (HDA), multiple displacement amplification (MDA), rolling circle amplification (RCA), and nucleic acid sequence-based amplification (NASBA). The term “isothermal amplification” refers to an amplification method that is performed without changing the temperature of the amplification reaction. Protons are released during an amplification reaction: for every deoxynucleotide triphosphate (dNTP) that is added to a single-stranded DNA template during an amplification reaction, one proton (HT) is released.

The term “sufficient amount” means an amount sufficient to produce a desired effect, e.g., an amount sufficient to modulate protein aggregation in a cell.

As used herein, a liquid sample can refer to a biological sample. A “biological sample” is a sample containing a quantity of organic material, e.g., one or more organic molecules, such as one or more nucleic acids e.g., DNA and/or RNA or portions thereof, which can be taken from a subject. As such, a “biological sample assay” is test on a biological sample which is performed to evaluate one or more characteristics of the sample. In some aspects, a biological sample is a nucleic acid amplification sample, which is a sample including or suspected of including one or more nucleic acids or portions thereof which can be amplified according to an embodiment.

A biological sample can be provided by a subject and include one or more cells, such as tissue cells of the subject. As used herein, the term “tissue” refers to one or more aggregates of cells in a subject (e.g., a living organism, such as a mammal, such as a human) that have a similar function and structure or to a plurality of different types of such aggregates. Tissue can include, for example, organ tissue, muscle tissue (e.g., cardiac muscle; smooth muscle; and/or skeletal muscle), connective tissue, nervous tissue and/or epithelial tissue. Tissue can, in some versions, include cells from the inside of a subject's cheek and/or cells in a subject's saliva. In some embodiments, a liquid sample is contemplated where a biological sample is referenced herein.

In some embodiments, the biological sample is swab of bodily fluid (mucous, vaginal fluid, seminal fluid, urine, saliva, etc.). In some embodiments, the biological sample is a tonsil/throat swab. In some embodiments, the biological sample is a nasal swab. In some embodiments, the biological sample is a nasopharyngeal swab. In some embodiments, the biological sample is a vaginal swab.

As noted above, a biological sample can be provided by a subject. In certain embodiments, a subject is a “mammal” or a “mammalian” subject, where these terms are used broadly to describe organisms which are within the class mammalia, including the orders carnivore (e.g., dogs and cats), Rodentia (e.g., mice, guinea pigs, and rats), and primates (e.g., humans, chimpanzees, and monkeys). In some embodiments, the subject is a human. The term “humans” can include human subjects of both genders and at any stage of development (e.g., fetal, neonates, infant, juvenile, adolescent, and adult), where in certain embodiments the human subject is a juvenile, adolescent or adult. While the devices and methods described herein can be applied in association with a human subject, it is to be understood that the subject devices and methods can also be applied in association with other subjects, that is, on “non-human subjects.”

A liquid sample (e.g., biological sample) can include, for example, human saliva, urine, human mucus, blood, an oral rinse, or a solid tissue such as buccal tissue. The biological sample can also include viruses, bacteria and/or spores.

In some embodiments, the biological sample is mixed with a preparation solution. In some embodiments, the preparation solution comprises a nucleic acid amplification preparation solution. In some embodiments, the preparation solution comprises an elution buffer. In some embodiments, the preparation solution alternatively or additionally includes optical property modifying reagents. According to some embodiments, an optical property of an optical property modifying reagent is changed due to the presence or the absence of a particular marker in a biological sample when the biological sample or one or more aspect thereof, are exposed to the optical property modifying reagent. Examples of optical properties that can change include color and absorbance as measure by spectrophotometry. Changes in optical properties can be detected and used to identify properties of the biological sample. In some embodiments, lysing cells of the sample with a lysing agent of the preparation solution and/or extracting nucleic acids therefrom. Such extracted nucleic acids can be released into a resulting prepared sample solution. In some embodiments, a step of extracting genomic deoxyribonucleic acid (DNA) from a biological sample is included. Where desired, the preparation solution is a nucleic acid amplification preparation solution and exposure to the solution prepares nucleic acids of the sample for amplification, e.g., isothermal amplification.

Also, as used herein, the phrase “optical property,” refers to one or more optically-recognizable characteristics, such as a characteristic resulting from wavelength and/or frequency of radiation, e.g., light, emitted by or transmitted through a sample, prior to, during, or following an assay reaction carried on using said sample, such as color, absorbance, reflectance, scattering, fluorescence, phosphorescence, etc. As such, modifying an optical property refers to changing such a characteristic.

### Systems

Aspects of the subject disclosure include systems for performing biological assays by modifying optical properties of biological samples and detecting these modified properties. Such systems can include a variety of devices, including a sample preparation device, and an assay device.

In some embodiments, the sample preparation device is configured to prepare a biological sample prior to being dispensed into the assay device. In some embodiments, the biological sample is collected using a sample collector (e.g., nasal swab, etc.). In some embodiments, the biological sample is mixed with a preparation solution to form a sample solution. In some embodiments, the preparation solution comprises a nucleic acid amplification preparation solution. In some embodiments, the preparation solution comprises an elution buffer. In some embodiments, the preparation solution comprises an optical property modifying reagent. In some embodiments, an optical property of an optical property modifying reagent is changed due to the presence or the absence of a particular marker in a biological sample when the biological sample or one or more aspect thereof, are exposed to the optical property modifying reagent. In some embodiments, the sample solution is configured to be dispensed into the assay device.

In some embodiments, the assay device comprises one or more thermal mixing modules, and/or one or more optical property modifying modules. In some embodiments, the thermal mixing module and the optical property modifying module are fluidly connected via a wax valve channel therebetween. In some embodiments, the biological assay is performed on a biological sample received by the thermal mixing module (for e.g., the biological sample may be provided in a sample solution). In some embodiments, as described herein, the assay device is configured to incubate the biological sample for a prescribed period of time before being transferred to the optical modifying module for carrying out one or more reactions.

In some embodiments, a thermally sealed valve located within the valve channel provides a barrier to prevent the biological sample (and corresponding sample solution) from flowing to the optical modifying module. In some embodiments, the thermally sealed valve is configured to soften, melt, and/or dissolve after receiving sufficient heat, such that the sample solution will be able to push through the valve and flow across the channel into the optical property modifying module. In some embodiments, such incubation of the sample allows for mixing the sample with a lytic agent, wherein such incubation and mixing helps lyse the cells and release biological material, as described herein, to help promote reactions in the optical modifying module. In some embodiments, the mixing of the sample with the lytic agent is via thermal mixing. The thermally sealed valves described herein may also be used in alternative mixing chambers that do not involve cell lysis (e.g., sample elution with reagents).

In some embodiments, the optical property modifying module comprises one or more reaction chambers, one or more of which can optionally have assay reagents (e.g., optical property modifying reagents, nucleic acid amplification reagents, or both) therein to mix and react with the sample in the prepared sample solution. In some embodiments, the reaction (e.g., amplification reaction), provides a reaction product, which reacts with optical property modifying reagent to generate a detectable change in the optical property of the biological sample, to indicate the presence, absence, or amount of analyte suspected to be present in the sample.

In some embodiments, assay reagents are present in the preparation solution. In some embodiments, assay reagents are present in the incubation chamber. In some embodiments, assay reagents are present in the one or more reaction chambers.

In some embodiments, each reaction chamber can include a sample receiving opening for receiving a biological sample from the sample inlet and/or a conduit. A sample receiving opening can be operatively, e.g., fluidically, connected to a sample inlet. In some versions, each reaction chamber includes one or more, e.g., two, additional openings, such as a “vented” and “supplementary,” or “first” and “second” opening. In some embodiments, the channel downstream from the wax valve channel 110 leads to one or more conduits that branch off the channel (e.g., like a spoke) and fluidically connect the channel to the reaction chamber. In some embodiments, each conduit connects to a different reaction chamber.

In some embodiments, each reaction chamber comprises a selective venting element as described herein. These selective venting elements are configured to allow any gas (e.g., air) and/or vapor to be discharged from the reaction chambers as the sample solution is entering the respective reaction chambers. Once a selective venting element is made impermeable to fluid, the methods include preventing further flow of fluid though a device or preventing fluid from flowing back out of the reaction chamber once it contacts the selective venting element, making it impermeable to fluid. As such, after such flow is stopped, diffusion is the only method for transporting any contaminants in and/or out of the reaction chamber. Therefore if the inlet and/or conduits have a sufficient length, the contaminant diffusion time is substantially longer than the reaction and/or readout time and a result is not affected by contaminants.

When miniaturizing and automating biochemical protocols into microfluidic systems, one challenge is how a sample can be accurately aliquoted into multiple reaction chambers while minimizing cross contamination. In some embodiments, this is accomplished by using multiple reaction chambers at the end of conduits that branch off the main fluid channel. Before reactions can take place in the chambers, the aliquots have to be isolated so that there is no cross talk between the reactions. In some embodiments, sample isolation can be achieved by placing input and/or output valves positioned between each chamber. The valves seal the chamber off from any cross talk. Although using multiple valves works to some extent, such a protocol imposes requirements to actively control the opening and closing of the valves, which in return requires energy, infrastructure, and expense to implement, and thus complicating the system design of the OPM module. Also, some valve structures work best when primed and as such, require the microfluidic system to be filled with an initial priming liquid. Such a priming step in turn complicates the system workflow. As such, according to versions of the subject methods, the methods do not include priming.

In contrast, the subject devices and methods can sufficiently provide automatic fluid flow control by passive aliquoting through one or more portions of a device such that an assay can be performed. For example, one or more fluids, e.g., air and/or biological sample, can be moved and/or prevented from moving through one or more portions of a device with little or no specific user interaction. Passive sealing of the device or portions thereof eliminates the need for active control and minimizes the complexity of the full device and the user steps required to run the device. As such, the subject disclosure provides simple and easy to use assay devices. Such subject devices and methods do not require valves or complicated valve control protocols. As such, the subject disclosure provides a simple and robust implementation of an on-chip aliquoting function with no moving parts. According to the subject embodiments, aliquot volumes and numbers are controlled by channel and chamber geometries (e.g., the length and cross-section of channels between the sample receiving openings of the reaction chambers being sufficiently long and sufficiently small, respectively, to prevent cross talk between contents of the reaction chambers). For example, in some embodiments, the cross-sectional area of each of the reaction chamber channels is no more than 1/10^{th} of the reaction chamber.

In some embodiments, the assay device comprises a substrate. Substrates can include one or more control unit, e.g., a central processing unit (CPU) or a field-programmable gate array (FPGA). Such a unit can include a memory and/or a processor, e.g., a microprocessor, configured to generate one or more outputs, e.g., electrical signals, based on one or more sets of inputs, e.g., inputs from a user and/or a sensor, and/or a timer, and/or instructions stored in the memory. A device can also include a user interface for receiving an input and operatively coupled to the control unit. In some embodiments, the substrate includes a printed circuit board (PCB), that is disposed below the thermal mixing module, the channel, and/or the optical property modifying module. In some embodiments, the substrate includes one or more heaters to supply heat to the assay device. Substrates can also include one or more thermal gap pads, for example, to couple heating elements on the PCB to appropriate fluidic locations.

In some embodiments, the sample preparation device is configured to be operatively couplable with the assay device.

By “operatively coupled,” “operatively connected,” and “operatively attached” as used herein, is meant connected in a specific way that allows the disclosed devices to operate and/or methods to be carried out effectively in the manner described herein. For example, operatively coupling can include removably coupling or fixedly coupling two or more aspects. Operatively coupling can also include fluidically and/or electrically and/or mateably and/or adhesively coupling two or more components. As such, devices which are operatively couplable are devices which are capable of being operatively coupled. Also, by “removably coupled,” as used herein, is meant coupled, e.g., physically and/or fluidically and/or electrically coupled, in a manner wherein the two or more coupled components can be un-coupled and then re-coupled repeatedly.

### Assay Devices

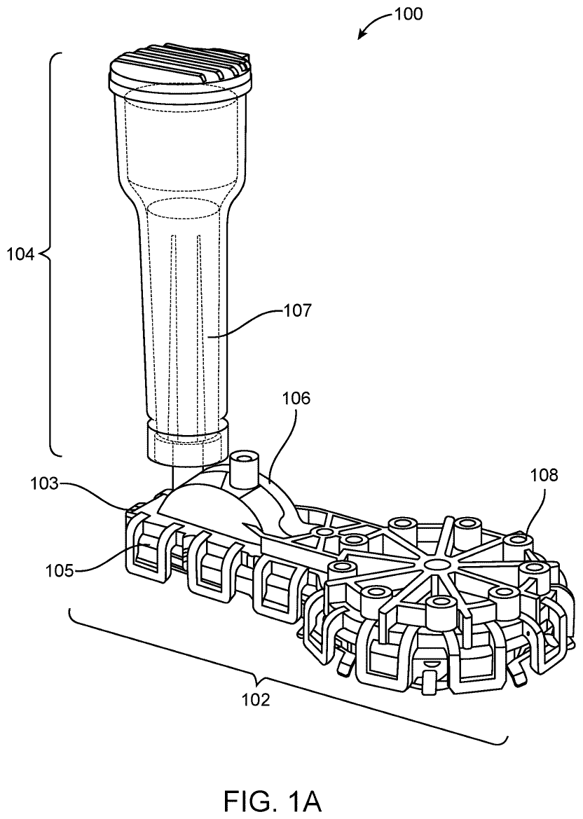

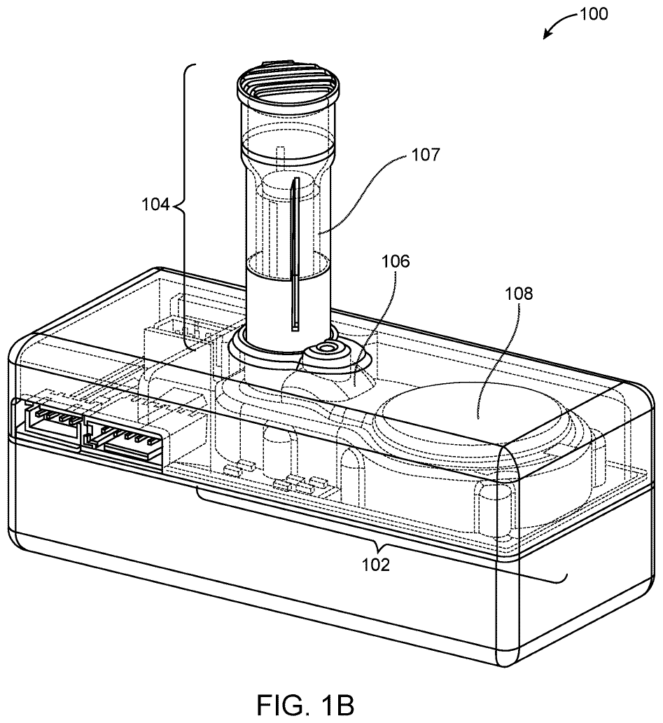

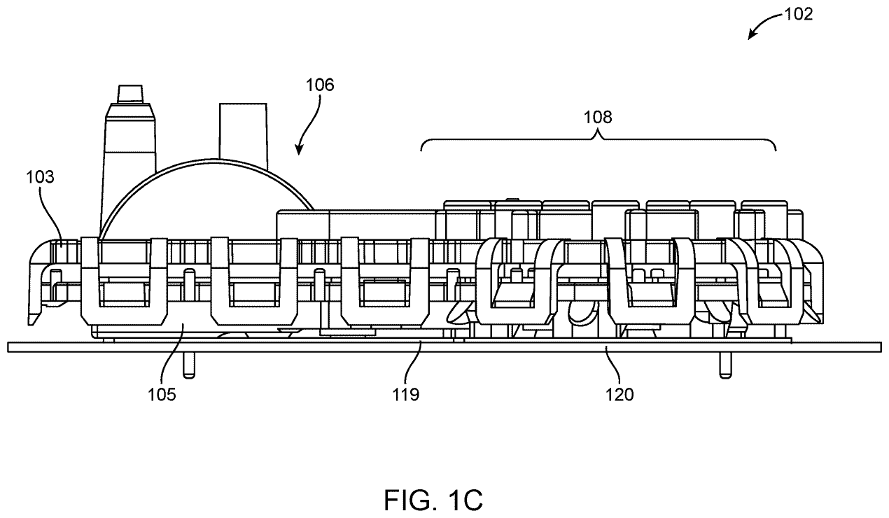

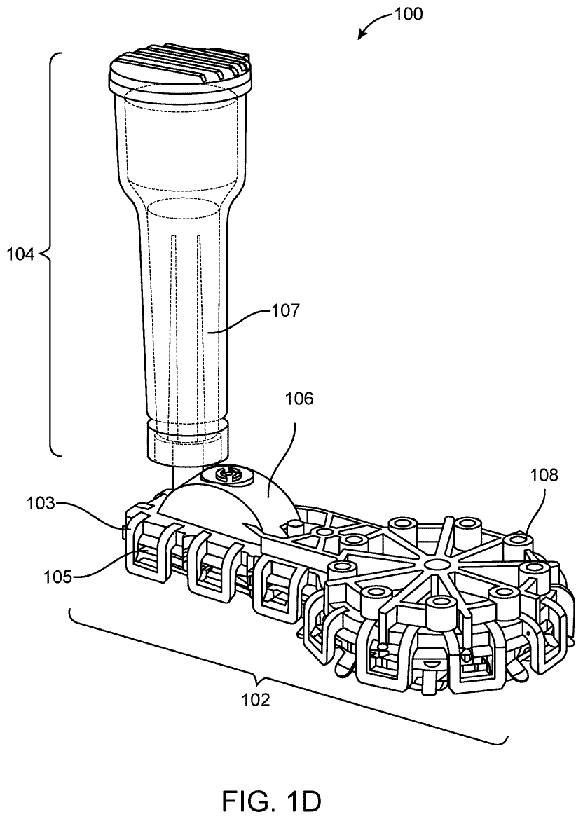

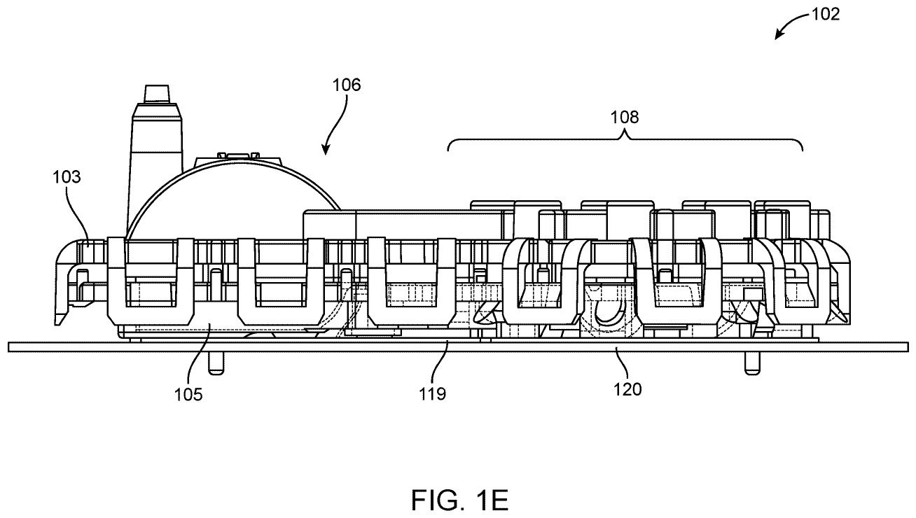

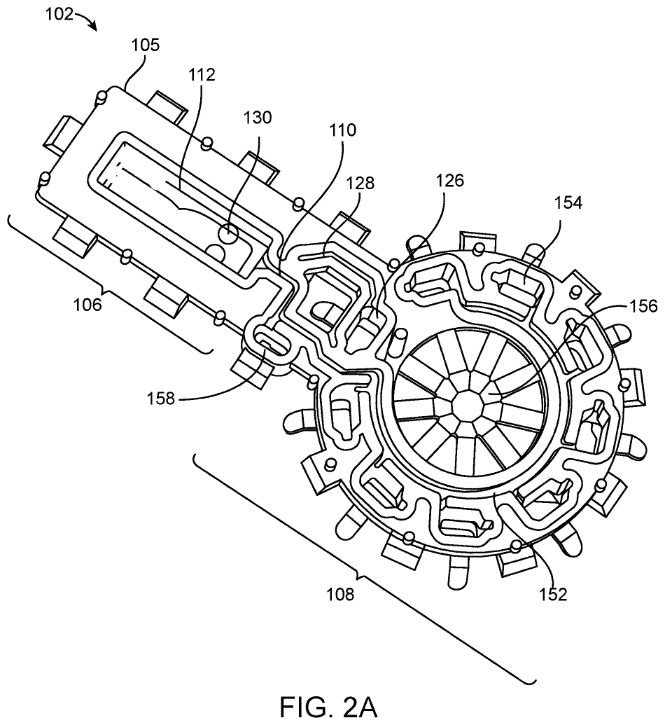

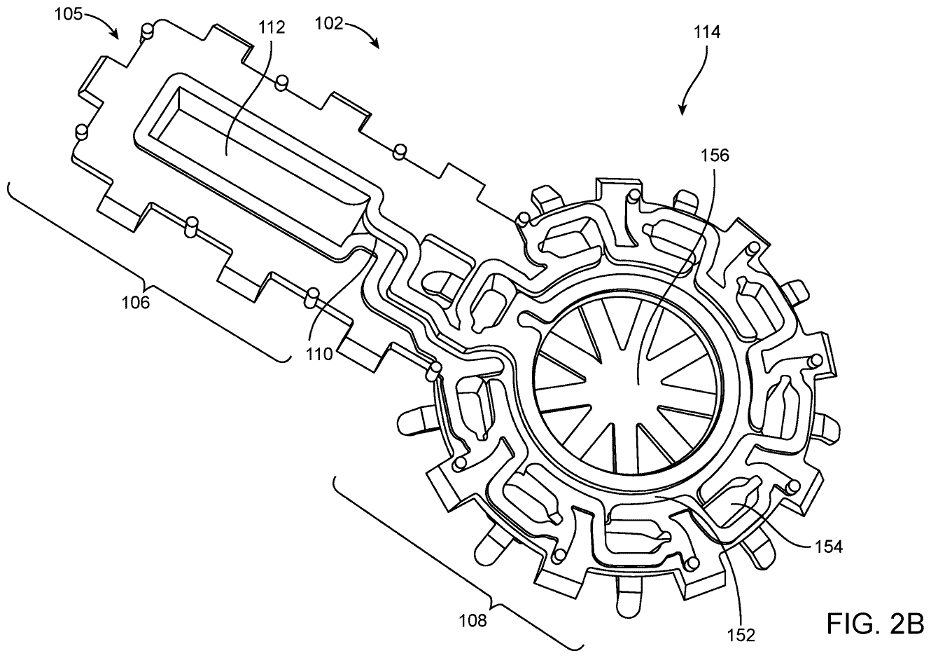

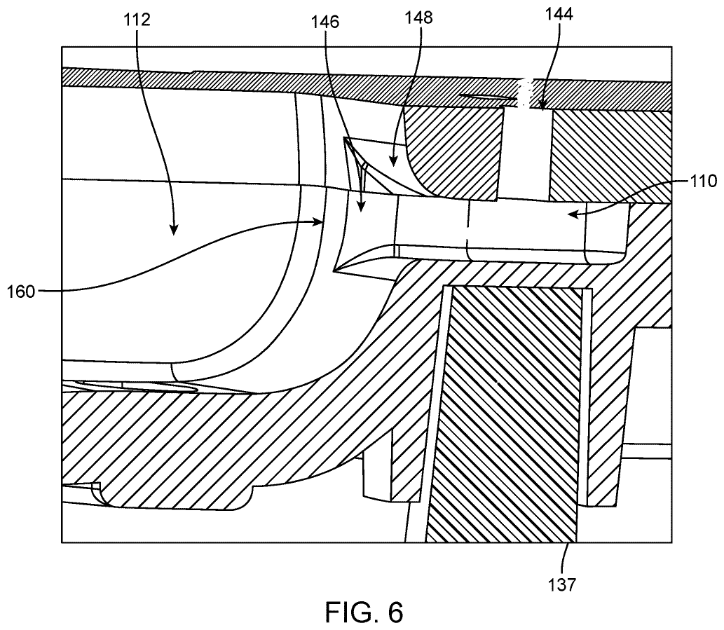

FIGS. 1A and 1B depict an exemplary embodiment of a system 100 described herein for performing a biological assay on a biological sample. In some embodiments, the system comprises an assay device 102 coupled with a sample preparation device 104. As used herein, “assay device” and test cartridge can be used interchangeably. In some embodiments, the assay device comprises a thermal mixing module 106 and optical property modifying (“OPM”) module 108, as described herein. In some embodiments, the sample preparation device 104 is used to elute the biological sample to make a sample solution 107. In some embodiments, the thermal mixing module 106 and the OPM module 108 are formed as a single unitary piece on the assay device 102. In some embodiments, the assay device 102 comprises a top portion 103, and a bottom portion 105, configured to be operatively coupled. In some embodiments, the top 103 and bottom portions 105 are coupled along a parting line of the assay device (see for e.g., 160 in FIG. 6). In some embodiments, the top and bottom portions of the device may sandwich a gasket made of an elastomer or other soft material to form a fluidic seal. FIG. 1C depicts a side view of an exemplary embodiment of an assay device 102. See also FIGS. 1D and 1E, which have a flat or low profile vent on the incubation chamber of the thermal mixing module 106. FIG. 2A depicts a top view of the bottom portion 105 of an embodiment of an exemplary assay device 102. As described herein, in some embodiments, the assay device 102 comprises a thermal mixing module 106 having an incubation chamber 112 therein, an OPM 108, and a channel 110 disposed therebetween. The term “incubation chamber” can be used interchangeably with “mixing chamber” herein. FIG. 2B depicts a similar assay device 102 without a sequestration chamber 158 and fill detection chamber 126.

In some embodiments, as shown in FIG. 1C, the assay device 102 further comprises a substrate 119 operatively coupled with a bottom portion 105 of the thermal mixing module 106, the channel 110, and the OPM module 108. In some embodiments, as described herein, the substrate 119 comprises a PCB 120. In some embodiments, the substrate 119 includes one or more heaters, each in operative communication with a power supply (for example, a single power supply can provide power to all heaters, or one or more power supplies can provide power to the heaters). In some embodiments, where there are multiple heaters, only one heater is powered at a time. In some embodiments, as described herein, the substrate 119 includes a mixing heater aligned with the thermal mixing module 106, a valve heater aligned with the channel 110, and/or a reaction heater aligned with the OPM module 108. In some embodiments, the substrate 119 comprises one or more thermal gap pads, for example, to couple heating elements on the PCB to appropriate fluidic locations.

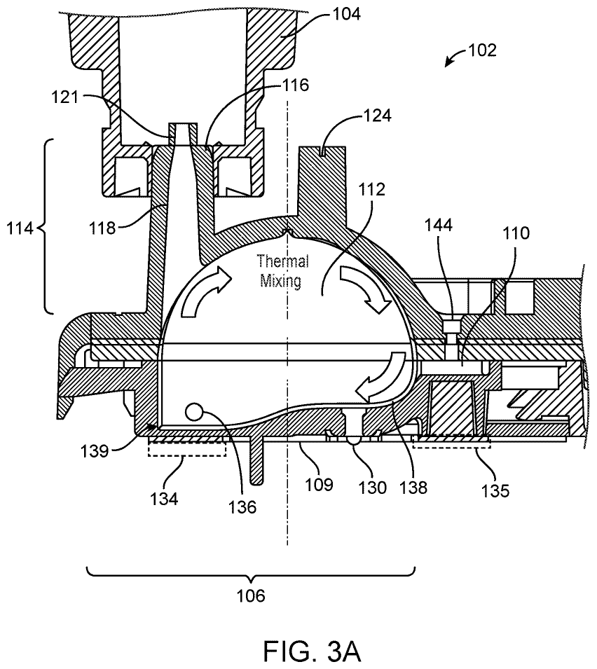

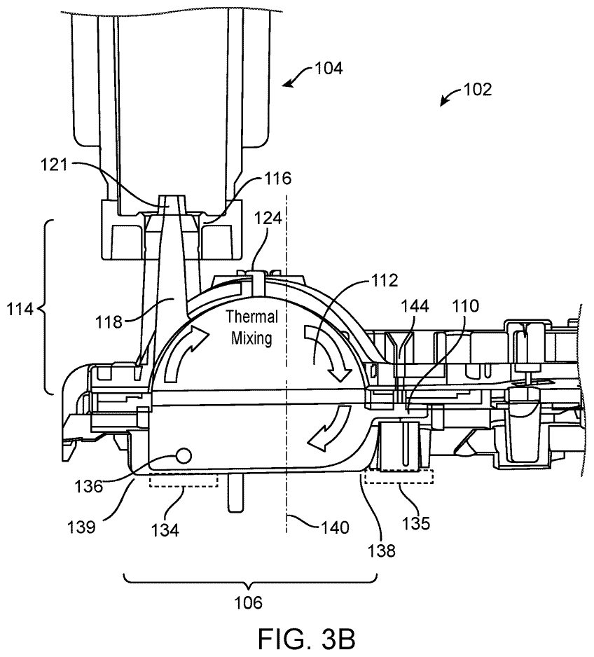

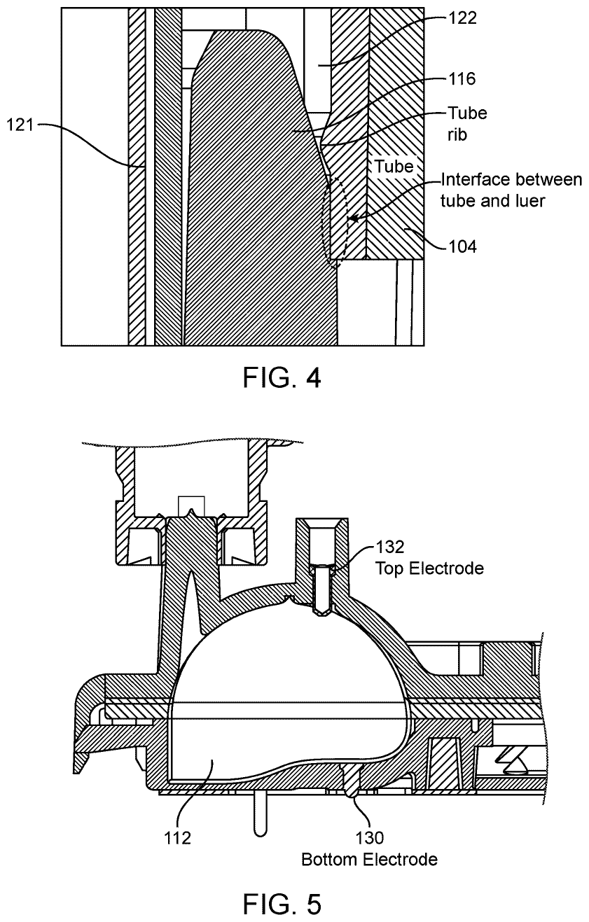

With reference to FIGS. 3A and 3B, side cutaway images of the assay device 102 are depicted, according to embodiments described herein. In some embodiments, the thermal mixing module 106 comprises a sample receiving module 114 and an incubation chamber 112. In some embodiments, the sample receiving module 112 is configured to be operatively coupled with a sample preparation device 104 to receive a sample therefrom (as described herein). In some embodiments, the sample receiving module 112 comprises a luer 116 defining an opening to a sample inlet 118 therein that is in fluidic communication with the incubation chamber 112. In some embodiments, the luer 116 is configured to operatively couple with the sample preparation device 104 to facilitate receipt of the sample into the assay device, e.g., through or around the puncturing element. For example, FIGS. 3A and 3B provide an exemplary coupling of the sample preparation device 104 to the luer 116. The sample preparation device is contemplated as a tube, vial, syringe, or other container. In some embodiments, a puncturing element 121 is configured to break a breakable seal of the sample preparation device to enable fluid flow therefrom. As used herein, “breakable seal” refers to a means for sealing an opening at an end of the sample receiving module that can be broken. Other means of breaking the breakable seal are contemplated in the devices of the present disclosure, e.g., the breakable seal can be pierced, impaled, penetrated, poked, sliced, cut, etc. With reference to FIG. 4, an exemplary depiction of the interaction between the luer 116 and the sample preparation device 104 is shown, wherein the luer 116 is configured to mate with a collar 122 of the sample preparation device, thereby forming a leak tight interface. The collar 122 can further include a tube rib that results in a stronger seal between the luer 116 and the collar 122. In some embodiments, the sample receiving module further comprises a puncturing element 121 to break the breakable seal of the sample preparation device 104 to enable fluid flow therefrom. In some embodiments, as described herein, the incubation chamber 112 is configured to receive the sample solution from the sample receiving module.

The puncturing element breaks the breakable seal, thereby placing the sample preparation device 104 in fluidic connection with the assay device or with the incubation chamber. In certain embodiments, the puncturing element provides for minimal deformation of the breakable seal and/or puncturing element when the puncturing element pierces the breakable seal. PCT/US2021/049178, the relevant disclosures of which are herein incorporated by reference, describes a bubble-free puncture mechanism that can be used for the breaking of the breakable seal allows for a clear puncture of the breakable seal with repeatable geometry, thereby minimizing the size and/or number of bubbles or preventing bubble formation entirely. As used, the term “bubble-free puncturing” refers to a mechanism that reduces (minimizes) the size and/or number of bubbles or prevents bubble formation entirely when the puncturing of, for example, the sample collection tube, occurs. The bubble-free free puncturing mechanism(s) described herein minimize or prevent the introduction of bubbles into the chamber (e.g., sample collection tube) that is being punctured.

The “seal” referenced in “breakable seal”, can be a layer of material, such as a polymeric and/or metallic material as such materials are described herein. In some embodiments, a seal is a foil sheet composed of aluminum and/or other metals.

In some embodiments, the sample preparation device comprises a valve rather than a breakable seal to be punctured. Examples of sample preparation devices comprises a breakable seal or valve are provided in PCT/US2017/022304, the relevant disclosures of which are herein incorporated by reference.

In some embodiments, the assay device can also include one or more filter for filtering fluid discharging through any part of the assay device or fluid discharging from the sample preparation device. A filter can be configured to filter a sample fluid prior to discharging the sample fluid through the valve.

In some embodiments, the interface comprises a hydrophobic material. In some embodiments, the puncturing element comprises a hydrophilic material. In some embodiments, the outlet port comprises a hydrophobic material. In some embodiments, the breakable seal comprises a hydrophilic material. In some embodiments, the interface comprises a plastic material. In some embodiments, the puncturing element comprises a metallic material. In some embodiments, the outlet port comprises a plastic material. In some embodiments, the breakable seal comprises a metallic material. In some embodiments, the puncturing element comprises a material having a hardness that is at least 2X times the hardness of a material comprising the breakable seal, such that the puncturing element provides for a clear puncture of the breakable seal with repeatable geometry and without deformation of the puncturing element. In some embodiments, the puncturing element has a hardness that is at least about 1X, about 2X, about 3X, about 4X, about 5X, about 6X, about 7X, about 8X, about 9X, about 10X, about 11X, about 12X, about 13X, about 14X, about 15X or about 20X times the hardness of the material comprising the breakable seal.

In some embodiments, an end of the second portion of the puncturing element that is configured to contact the breakable seal comprises a blunt tip, such that a surface area of the end of the second portion is orthogonal to the length of the puncturing element. In some embodiments, an end of the second portion of the puncturing element that is configured to contact the breakable seal comprises a sharp tip.

### Incubation Chamber Vent

In some embodiments, the incubation chamber 112 includes a vent 124 configured to allow any gas (e.g., air) and/or vapor to be discharged from the incubation chamber 112 as the sample solution is entering the incubation chamber 112. In some embodiments, the vent 124 is open to the surrounding environment, wherein upon filling the incubation chamber 112, the sample solution overflows the top of the vent 124. In some embodiments, the vent 124 comprises a selective venting element configured to seal upon direct contact with liquid, preventing the liquid (e.g., sample solution) from passing therethrough.

Selective venting elements can be porous and as such, have a plurality of pores extending therethrough. Such elements can have a passively tunable porosity and/or can control flow of one or more fluids, e.g., gas, such as air and/or liquids, such as a biological sample, within a device. For example, in some embodiments, the vent 124 comprises a self-sealing sintered polymer vent plug or a hydrophobic porous membrane. In some embodiments, a selective venting element is a porous hydrophobic membrane that has pores that allow gases (e.g., air) therethrough but not droplets of water or other polar liquids.

The phrase “passively tunable porosity,” as used herein, refers to the ability of having a first conformation in which one or more gasses, e.g., air, can pass therethrough, e.g., through pores, and a second conformation in which fluids including the one or more gasses and liquids, such as liquids including a biological sample, are prevented or reduced from passing therethrough, e.g., through the pores, and proceeding automatically from the first to the second conformation upon contact with a liquid. Also, in the second conformation, the selective venting elements prevent evaporation of the liquids therethrough, e.g., through the pores. Furthermore, in the second conformation, the selective venting elements can fluidically seal a fluidic passage, e.g., a reaction chamber at an end by covering an opening of the reaction chamber, e.g., a venting opening, and prevent passage of fluid, including evaporation, therethrough. In addition, selective venting elements are configured to proceed from the first conformation to the second conformation passively, e.g., automatically without user interaction, upon contacting one or more liquids, such as liquids including a liquid sample, with the selective venting elements or a portion thereof, e.g., a surface, such as a surface forming a wall of the incubation chamber or reaction chamber. As such, in some embodiments, selective venting elements can be self-sealing to liquids and gasses when contacted by a liquid.

The use of the selective venting elements can minimize or eliminate the need for input and/or output valves within the device (or device channels). Although using multiple valves works to some extent, such a protocol imposes requirements to actively control the opening and closing of the valves, which in return requires energy and infrastructure to implement, and thus complicating the system design. Passive sealing of the device or portions thereof eliminates the need for active control and minimizes the complexity of the full device and the user steps required to run the device. The use of at least one selective venting element in each of the reaction chambers can facilitate passive control over fluid flow within the device; and thus, in some embodiments, a valve is not required for the device to function and/or to control fluid flow to the chambers (e.g., reaction chambers).

With reference to FIG. 2A, in some embodiments, the assay device comprises a fill-detection chamber 126 in fluidic communication with incubation chamber 112 via a sensing channel 128, optionally wherein the fill-detection chamber 126 is operatively coupled with a light pipe that is operatively connected to an LED. For example, in some embodiments, the sensing channel 128 is in fluidic connection with the incubation chamber 112 such that as fluid enters the incubation chamber 112 it also flows into the fill-detection chamber via the sensing channel 128. An opening at a terminus of the sensing channel can allow fluid communication between the sensing channel 128 and incubation chamber 112. In some embodiments, the fill-detection chamber 126 includes (or is operatively connected to) a light sensor to detect the light from the light pipe, wherein filling of the fill-detection chamber 126 with the sample solution (via filling of the incubation chamber 112) results in the light sensor detecting a decrease in light (due to the sample, which may be dark depending on the liquid (e.g., buffer) used). The position of the opening at the terminus of the sensing channel 128, connecting the sensing channel 128 to the incubation chamber 112, can determine when liquid filling is detected and the resulting output (e.g., activation of heating and/or stirring). For example, the opening of the sensing channel 128 can be placed at the top of the incubation chamber 112, such that liquid only begins entering the fill-detection chamber 126 when the incubation chamber 112 is at or near maximum liquid capacity. Then, the liquid flows into the fill-detection chamber 126, thereby attenuating the light detected by the light sensor.

In some embodiments, the light sensor comprises one of a complementary metal-oxide semiconductor (CMOS) chip, a photodiode, a phototransistor, a photocell, and a photomultiplier tube.

### Incubation Chamber Liquid Detection

In some embodiments, the assay device includes one or more sensors configured to detect i) entry of liquid (e.g., the sample solution) into the incubation chamber (e.g., initial entry of the sample solution), ii) a liquid filled incubation chamber, or iii) both. In some embodiments, the one or more sensors operate as an interlock and help prevent activating a heater (e.g., mixing heater) prior to liquid entering the incubation chamber. For example, prematurely activating the mixing heater prior to the sample solution entering the incubation chamber 112 may overheat the assay device 102, and/or unnecessarily reduce a battery life of a power supply providing power to the mixing heater. In some embodiments, a sensor is operatively coupled with a power supply for activating a heater. For example, in some embodiments, the substrate 119, which can comprise a PCB, and optionally, a thermal gap pad, is in operative communication with a controller and a power supply, wherein the controller will allow the power supply to supply power to the mixing heater once the one or more sensors detects fluid and/or a prescribed fluid level within the incubation chamber 112.

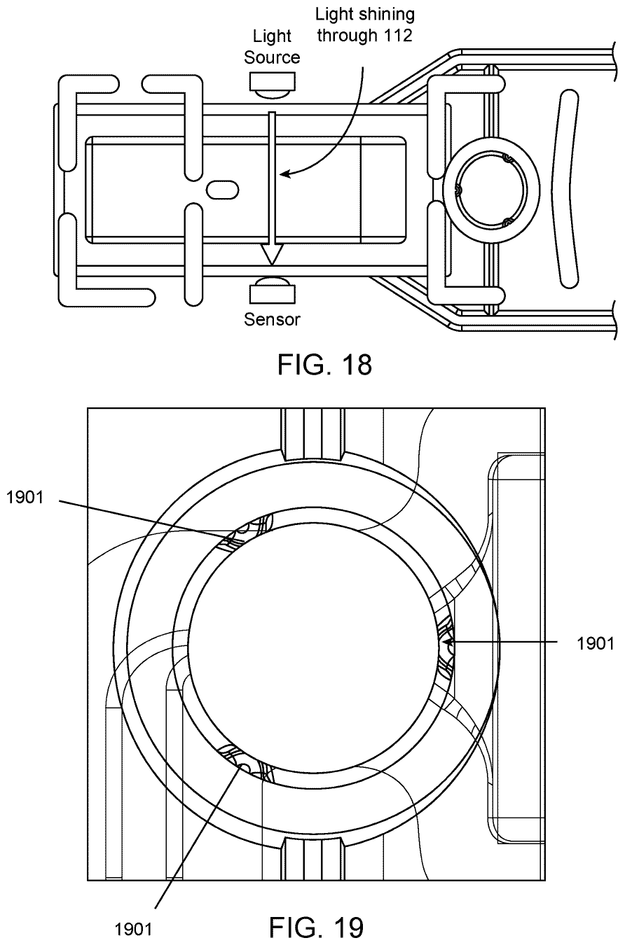

In some embodiments, the incubation chamber can include one or more light sources configured to emit light. Such light sources can be operatively coupled to the one or more sensors and/or the PCB and/or a controller/control unit such that the sensor is positioned (e.g., across from the light source within the same chamber) to receive/detect light from the light source. See, e.g., FIG. 18. In some embodiments, the one or more sensors can be configured to detect an optical property, e.g., a wavelength of light, e.g., color, and/or a change in an optical property, such as a wavelength of light emitted from contents of a chamber. In some embodiments where the light source and one or more sensors are within the same chamber (e.g., incubation chamber, reaction chamber, fill detection chamber, etc.), the light source is emitting light prior to entry of liquid into the chamber and as liquid enters and fills the chamber, the light signal detected by the sensor (e.g., light sensor) is attenuated. Once the light signal has attenuated to a threshold amount, an input from the sensor indicates that liquid is present in the chamber. In some embodiments, the controller can respond by generating an output that initiates an action (e.g., activating a heating element or mixing element). Light sources according to various embodiments can also include one or more light emitting diode (LED). In some embodiments, this is also accomplished with the use of one or more light pipes, wherein the one or more light pipes enables light to be transmitted between to the sensor. In some embodiments, the one or more light pipes are operatively connected to the incubation chamber and it transmits light shining through the chamber (e.g., incubation chamber, reaction chamber, fill detection chamber, etc.) to one or more sensor. In some embodiments, the light emitted by the light source is transmitted through the one or more light pipes using at least one of one or more reflecting surfaces and/or one or more refracting surfaces located within the one or more light pipes.

In some embodiments, once fluid is detected in the incubation chamber, the system initiates the incubation heating step. An indicator on the display can then signal that sample processing has begun. If fluid is not detected, the system can stay in a “ready to load sample” state (e.g., similar to the indicator used when the device was first powered on or loaded). This would enable the device to flag an error when reagents have been exposed to air for too long.

In some embodiments, the sensors will provide a signal indicating fluid detection within the incubation chamber 112, wherein the signal can be an audible signal (e.g., a beeping noise, which may originate from the PCB), and/or a visual signal (e.g., a flashing light).

In some embodiments, the one or more sensors comprises a capacitive sensor disposed below the incubation chamber 112. In some embodiments, the capacitive sensor is configured to measure an increased dielectric constant in the sample solution (e.g., which includes a buffer) as compared with air. In some embodiments, the capacitive sensor being located on the bottom (of the incubation chamber) is configured to detect sample solution entry into the incubation chamber 112 or sample solution level within the incubation chamber 112.

In some embodiments, the one or more sensors comprises a conductive sensor that comprises one or more electrodes. In some embodiments, the one or more electrodes are operatively coupled with the substrate (e.g., PCB), and configured to penetrate through a bottom wall of the incubation chamber, such that the electrode(s) are exposed within the incubation chamber (see for example electrodes 130 in FIGS. 2A, 3A, and 5). Accordingly, in some embodiments, the one or more electrodes 130 are configured to contact a liquid (e.g., sample) and therefore detect initial entry of the sample solution in the incubation chamber 112. In some embodiments, the electrodes 130 require a liquid so as to conduct an electrical charge between the two electrodes 130. For example, in some embodiments, the sample solution includes an ionic buffer that has ions configured to conduct electricity between the two electrodes 130. Accordingly, when there is no fluid in the incubation chamber 112, there may not be any conduction of electrical charge between the electrodes 130. In some embodiments, conduction of an electrical charge between the electrodes 130 enables activation of the mixing heater 134, via the PCB. In some embodiments, the electrodes 130 comprise metal, a polymer, or an elastomer with conductive particles embedded therein to make the whole electrode conductive.

With reference to FIG. 5, in some embodiments, in addition to or alternative to having an electrode 130 at a bottom of the incubation chamber 112, the assay device further comprises an electrode 132 located at a top of the incubation chamber. In some embodiments, similar to two electrodes located at the bottom of the incubation chamber, a bottom electrode 130 and a top electrode 132 will conduct an electrical charge therebetween when exposed to a liquid (e.g., sample). Accordingly, in some embodiments, a top 132 and bottom 130 electrode configuration enables to detect when the incubation chamber 112 is filled with liquid (thereby creating a conductive path through the incubation chamber height). In some embodiments, the top electrode 132 is operatively connected to the substrate (e.g., PCB) via a conductive clip or wire. In some embodiments, the mixing heater 134 will activate upon the incubation chamber being filled with the sample solution. In some embodiments, the assay device comprises two bottom electrodes 130, and a top electrode 132, so as to be able to detect initial liquid presence (or entry) in the incubation chamber 112, and a level of the liquid (e.g., sample) in the incubation chamber 112.

In some embodiments, the one or more sensors comprises the light sensor as described herein, wherein a fill-detection chamber 126 is used to detect a fluid level in the incubation chamber 1112, such that a decreased detection of light by the light sensor indicates the incubation chamber being filled with a liquid (e.g., sample solution) (see FIGS. 2A and 18 for example). In some embodiments, such detection allows for activation of the mixing heater 134.

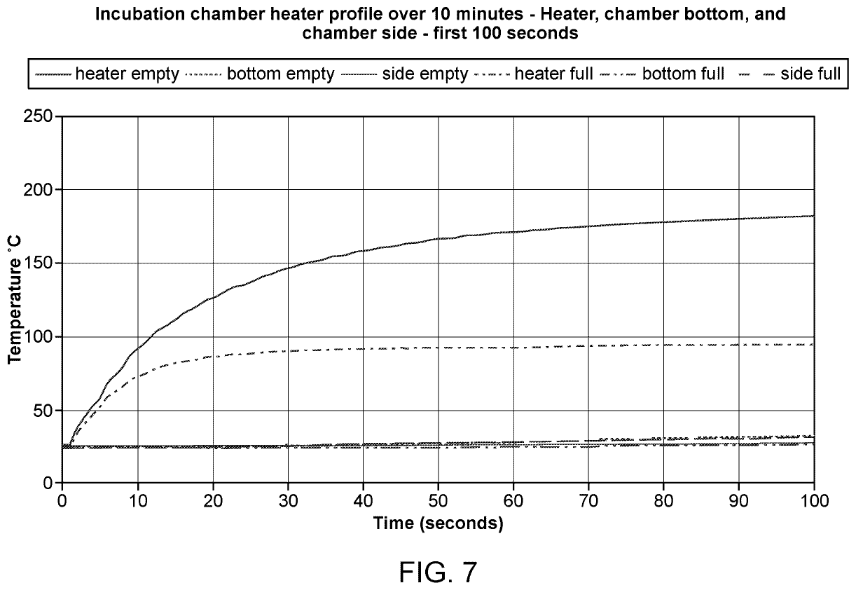

In some embodiments, the one or more sensors comprises a thermocouple operatively coupled with the mixing heater 134 and configured to measure the temperature of said mixing heater 134 or another area where the PCB is in close thermal contact with the incubation chamber. In some embodiments, a constant power supply is provided to the mixing heater 134 (by one or more power supplies as described herein), such that a delay in a temperature rise by the mixing heater 134, as detected by the thermocouple, indicates the presence of liquid within the incubation chamber 112. For example, in some embodiments, the sample solution includes a buffer having a high heat capacity, such that the mixing heater 134 temperature rises more slowly when said sample solution is present in the incubation chamber 1121 than when not. FIG. 7 provides an exemplary comparison of temperature rise of the mixing heater 134, a side wall, and a bottom wall of the incubation chamber 112, when the incubation chamber 112 is i) empty, and ii) filled with a buffer (e.g., provided with the sample solution). As shown, the temperature rise when the incubation chamber 112 is full is much slower when compared with an empty incubation chamber 112. In some embodiments, such detection in temperature rise by the thermocouple allows for activation of the mixing heater 134.

### Sample Solution Mixing

In some embodiments, the incubation chamber is configured to mix the sample solution with a lytic agent so as to lyse the cells obtained via the biological sample. In some embodiments, such lysing allows for nucleic acid within said cells to be released for amplification in the OPM module 108. In some embodiments, the lytic agent is disposed within the incubation chamber 112 prior to the sample solution being dispensed therein. In some embodiments, the lytic agent is provided to the incubation chamber 112 after the sample solution has been dispensed within the incubation chamber 112. In some embodiments, the lytic agent is dispensed substantially simultaneously with the sample solution into the incubation chamber 112. In some embodiments, the lytic agent is suspended in the sample inlet just past the puncturing element to promote faster and more complete mixing.

In some embodiments, the lytic agent comprises a lyophilized pellet 136 that is rehydrated upon exposure to the sample solution. In some embodiments, the lytic agent is a liquid. In some embodiments, the lytic agent comprises Dithiothreitol (DTT), Proteinase K, Mutanolysin, Lysotaphin, Lysozyme, or any combination thereof. In some embodiments, the lytic agent further comprises one or more surfactants, such as Tween. In some embodiments, the lytic agent further comprises one or more components of the buffer provided with the sample.

In some embodiments, where a lytic agent is used, an RNase inhibitor, or some other agent that protects against nucleic acid degradation, is also contemplated.

With reference to FIGS. 3A and 3B, in some embodiments, the sample solution is mixed with the lytic agent 136 via thermal mixing, as described herein. In some embodiments, the mixing heater 134 is configured to supply heat to the incubation chamber 112, so as to heat a portion of the sample solution located within the vicinity of the mixing heater 134 more rapidly (as compared to the remaining sample elsewhere in the incubation chamber 112), and thereby rise the temperature of said portion of the sample solution more rapidly. Such rise in temperature decreases the density of said portion of the sample solution, thereby increasing the buoyancy of said portion as compared to the surrounding sample solution, such that said portion of the sample solution rises within the incubation chamber 112, and causes another portion of the sample solution to move into its place within the vicinity of the mixing heater 134 (for example, see movement of arrows in FIGS. 3A and 3B representing approximate motion of sample solution). In some embodiments, continued supply of heat to the incubation chamber 112 progressively heats the sample solution. In some embodiments, said heating of the sample solution helps further lyse the cells within the sample solution.

In some embodiments, the incubation chamber is configured to mix a liquid sample, absent a lytic agent. In some embodiments, the incubation chamber is configured to mix a liquid sample and a reagent, as contemplated by one of ordinary skill in the art.

As depicted in FIGS. 3A and 3B, in some embodiments, the incubation chamber 112 is configured with one or more rounded edges 138 so as promote circulation of the sample solution therein, wherein such circulation is provided through movement of the fluid via the mixing heater 134. In some embodiments, said one or more rounded edges 138 helps the circulation of the fluid in an approximate circular motion. In some embodiments, a corner of the incubation chamber 112 within the vicinity of the mixing heater 134 may not be rounded 139.

In some embodiments, the dimensions of the incubation chamber 112 are prescribed so as to optimize sample solution mixing therein, and help reduce or minimize areas of dead volume wherein the sample solution may be more stagnant and thus have reduced mixing. In some embodiments, the length of the incubation chamber 112 is between 0.75 and 2 times the height of the chamber. In some embodiments, the width of the chamber is between ⅙^{th} and ⅔^{rd} the average of the channel length and width.

In some embodiments, the thickness of one or more walls of the incubation chamber 112 is prescribed so as to optimize sample solution mixing via shearing as the sample solution passes said walls. If the chamber is too narrow and the walls are too close together, the wall friction slows down mixing. The wider the walls are, the lower the fraction of the boundary walls relative to the overall cross-section, then shear stress caused by wall friction does not contribute significantly to mixing.

In some embodiments, the incubation chamber 112 has a volume from about 0.5 mL to 5 mL, from about 0.1 mL to about 100 mL, from about 0.5 mL to about 50 mL, from about 0.75 mL to about 25 mL, or from about 1 mL to about 10 mL.

As depicted in FIGS. 3A and 3B, in some embodiments, the mixing heater 134 is located offset from a center position 140 of the incubation chamber 112 (e.g., along a width of the incubation chamber). In some embodiments, positioning the mixing heater 134 offset from a center position 140 (e.g., along a width) of the incubation chamber 112 helps promote uneven buoyant mixing of the sample solution (as opposed to a more central location of heating). Accordingly, as shown in FIGS. 3A and 3B, the portion of the sample solution located within the vicinity of the mixing heater 134, wherein the incubation chamber may have a corner that is not rounded 139, will receive more heat than the sample solution elsewhere in the incubation chamber 112, so as to promote fluid motion, and thereby mixing, within the incubation chamber 112 through the difference in density (as described herein).

Various sizes are contemplated for the mixing heater. In some embodiments, the mixing heater is 1/50^{th} size of the incubation chamber. In various embodiments, the length of the mixing heater is no more than half the length of the incubation chamber. In some embodiments, the size of the mixing heater is no more than half the size of the incubation chamber.

In some embodiments, as described herein, the substrate comprises a controller in operative communication with a power supply that provides power to the mixing heater 134 for heating thereof. In some embodiments, the power supplied to the mixing heater 134 is regulated by the controller based on the temperature of the mixing heater 134, such that a sensor (e.g., a thermocouple) sends a feedback signal to the controller to vary the power supplied so as to maintain the mixing heater 134 at a prescribed temperature (e.g., between 30° C. and 65° C.). In some embodiments, the power supplied to the mixing heater 134 is at a constant or substantially constant rate, regardless of the temperature of the mixing heater 134. In some embodiments, such constant or substantially constate rate of power supply is sufficient since thermal mixing is promoted via a non-uniformity in temperature. In some embodiments, the mixing heater 134 is a resistive heater.

In some embodiments, a thermal gap pad 109 conducts heat from the PCB heaters to different parts of the device. In some embodiments, a metal element conducts heat from the PCB heaters to the thermally sealed valve and/or or the incubation chamber. In some embodiments, that metal element is cylindrical. In some embodiments, that metal element is an aluminum cylinder. The metal element can be made of any heat conductive metal, e.g., silver, copper, aluminum, or gold. In some embodiments, the metal element is a metal alloy, e.g., that comprises at least one heat conductive metal. In some embodiments, the metal element is press fit into position between the PCB and the chamber or device component to receive the heat (e.g., adjacent to the thermally sealed valve). In some embodiments, the metal element is press fit to crush ribs. As used herein, “crush ribs” refer to a set of very small projections that deform as a part is press fit into them. Without being limited to a mechanism of action, the use of press fitting (and optionally crush rib press-fitting) minimize the heat loss to the rest of the assay device and/or environment. FIG. 19 provides an illustration of an aluminum cylinder secured by three crush ribs 1901. The aluminum cylinder conducts heat from the PCB heaters to the thermally sealed valve.

As described herein, in some embodiments, the wax valve channel 110 serves as an outlet from the incubation chamber 112 to the OPM module 108. In some embodiments, the wax valve channel 110 includes a thermally sealed valve, as described herein that provides a barrier to prevent the sample solution from flowing through the channel 110, so as to keep the sample solution within the incubation chamber 112 for said mixing and/or incubation to occur for a prescribed amount of time. In some embodiments, for an incubation chamber having a volume of 1 mL, the prescribed amount of time for mixing and/or incubation (so as to lyse the cells within the sample solution) is from about 1 to 20 minutes, about 3 to 10 minutes, about 2 to 15 minutes, or about 5 to 10 minutes.

### Thermally Sealed Valve and Channel

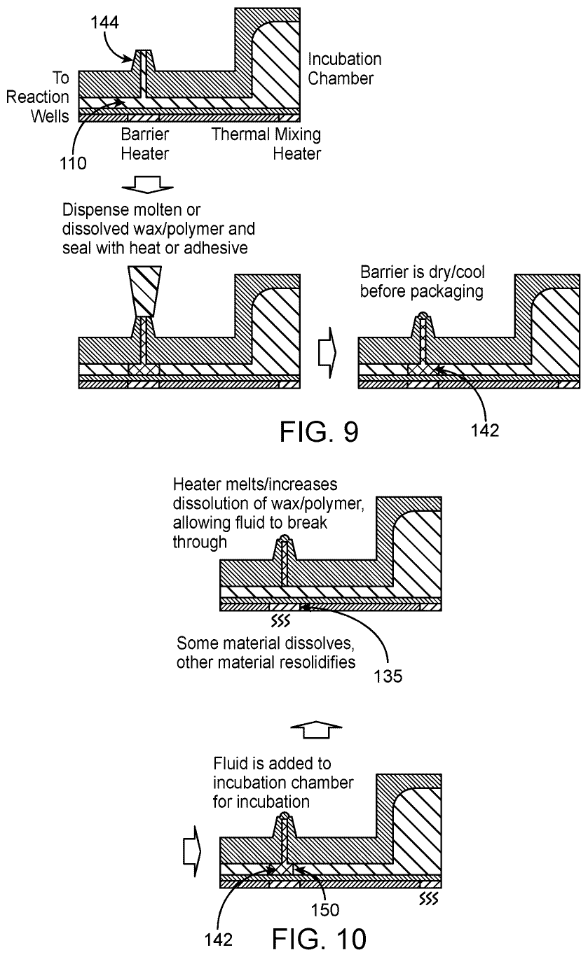

In some embodiments, as described herein, a wax valve channel 110 connects the thermal mixing module 106 with the OPM module 108. In some embodiments, the wax valve channel is provided with a valve to provide a barrier that helps prevent the sample solution from prematurely entering the OPM module 108 prior to sufficient mixing and lysing of cells in the sample solution. In some embodiments, said valve is a thermally sealed valve (e.g., see 142 in FIG. 9), which may be initially solid at a first temperature, but upon receiving sufficient heat, the wax valve softens, melts, and/or dissolves, thereby allowing liquid (e.g., the prepared sample solution, which is the sample solution after sufficient mixing and/or incubation) to pass through said valve to the OPM 108. In some embodiments, the thermally sealed valve comprises a water-soluble wax. In some embodiments, the thermally sealed valve comprises a polymer. In some embodiments, the polymer is water soluble. In some embodiments, the valve comprises a wax, optionally a water-soluble wax. In some embodiments, the thermally sealed valve comprises a polymer such as, for example, polyethylene glycol (PEG). In some embodiments, the wax (e.g., PEG) dissolves with the sample solution after softening and/or melting, thereby reducing or eliminating the risk of the wax re-solidifying when removed from a source of heat (and thus cooling). Accordingly, this helps prevents occlusion (either partially or fully) of flow within the wax valve channel 110 or farther downstream in the assay device. In some embodiments, the dissolved valve material has minimal or no deleterious effect on the sample solution with respect to reactions (e.g., amplification) within the OPM module 108.

In some embodiments, the thermally sealed valve comprises other types of waxes and material for use as thermally sealed valve material known in the art. Non-limiting examples of wax materials that can be used in devices of the present disclosure include polyethylene glycol (PEG), HYDROSOL™, Aquasol, and Cerita. In some embodiments, the wax can be a non-soluble wax and an emulsifier such as polysorbate.

In some embodiments, the actuation temperature of the thermally sealed valve is dependent on a molecular weight of the material thereof. In the context of thermally sealed valve material molecular weight, the term “about” also indicates that in preparations of polymer (e.g., PEG), some molecules will weigh more and some less, than the stated molecular weight. As used herein “actuation temperature” refers to the temperature at which the wax dissolves. For example, in some embodiments, the wax has a molecular weight of about 1,305 g/mol to about 10,000 g/mol, which corresponds to an actuation temperature range from about 43° C. to about 63° C. In some embodiments, increasing the molecular weight of the valve increases the actuation temperature. In some embodiments, the molecular weight of the valve is from about 500 g/mol to about 20,000 g/mol, from about 1,000 g/mol to about 15,000 g/mol, or about 1305 g/mol to about 10,000 g/mol. In some embodiments, the thermally sealed valve has a molecular weight from about 1,300 g/mol to about 10,000 g/mol. In some embodiments, the thermally sealed valve has a molecular weight of about 6,000 g/mol. In some embodiments, the thermally sealed valve material is PEG.

In some embodiments, the thermally sealed valve material is selected based on its ability to remain stable (including in a solid state) under room temperature and standard shipping conditions. For example, the higher the molecular weight, the higher shipping and/or storage temperatures that the thermally sealed valve material can withstand. Standard shipping condition requirements for devices are known to those of ordinary skill in the art. For example, the standard shipping conditions required could be based on ASTM D4332 conditions (e.g., ASTM D4332-14). Briefly, the ASTM D4332 test describes a plan for conditioning containers, packages, or packaging components. The purpose is so that the containers approach or reach equilibrium with the atmosphere to which they may be exposed. In some embodiments, the thermally sealed valve material must maintain stability (e.g., remain solid) up to about 60° C., and optionally about 15% relative humidity. In some embodiments, the thermally sealed valve material must maintain stability (e.g., remain solid) up to about 40° C., and optionally about 90% relative humidity. In some embodiments, the thermally sealed valve material must maintain stability at temperatures as low as about −30° C. In some embodiments, the thermally sealed valve material must maintain stability (e.g. remain solid) up to about 40° C., 41° C., 42° C., 43° C., 44° C., 45° C., 46° C., 47° C., 48° C., 49°° C., 50° C., 51° C., 52° C., 53° C., 54° C., 55° C., 56° C., 57° C., 58° C., 59° C., 60° C., 61° C., 62° C., 63° C., 64° C., or 65° C. In some embodiments, the thermally sealed valve material must maintain stability (e.g., remain solid) between about 40° C. and 60° C. or 40° C. and 65° C.

In some embodiments, the thermally sealed valve material must maintain stability (e.g., remain solid) at a relative humidity of about 15%. In some embodiments, the thermally sealed valve material must maintain stability (e.g., remain solid) at a relative humidity of about 90%. In some embodiments, the thermally sealed valve material must maintain stability (e.g., remain solid) at a relative humidity of about 15% and 90%. In some embodiments, the thermally sealed valve material must maintain stability (e.g., remain solid) at a relative humidity of about 15%, 20%, 25%, 30%, 35%, 40%, 45%, 50%, 55%, 60%, 65%, 70%, 75%, 80%, 85%, or 90%.

In some embodiments, the thermally sealed valve material must maintain stability (e.g., remain solid) at any of the temperatures and/or relative humidity conditions disclosed herein for at least 6 hours, 12 hours, 24 hours, 36 hours, 38 hours, or 72 hours. In some embodiments, the thermally sealed valve material must maintain stability (e.g., remain solid) at any of the temperatures disclosed herein for at least 6 hours. In some embodiments, the thermally sealed valve material must maintain stability (e.g., remain solid) at any of the temperatures disclosed herein for at least 12 hours. In some embodiments, the thermally sealed valve material must maintain stability (e.g., remain solid) at any of the temperatures disclosed herein for at least 24 hours. In some embodiments, the thermally sealed valve material must maintain stability (e.g., remain solid) at any of the temperatures disclosed herein for at least 36 hours. In some embodiments, the thermally sealed valve material must maintain stability (e.g., remain solid) at any of the temperatures disclosed herein for at least 48 hours. In some embodiments, the thermally sealed valve material must maintain stability (e.g., remain solid) at any of the temperatures disclosed herein for at least 72 hours.

In some embodiments, the thermally sealed valve material maintains stability (e.g., remains solid) up to 60° C. for 72 hours.

In some embodiments, a preferred material for the thermally sealed valve also has an actuation time averaging between about 30 seconds and 50 seconds. In some embodiments, the actuation time is no more than about 30 seconds. In some embodiments, the actuation time is no more than about 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, or 50 seconds. In some embodiments, the actuation time is between about 25 seconds and 30 seconds, 25 seconds and 35 seconds, 25 seconds and 40 seconds, 25 seconds and 45 seconds, or 25 seconds and 50 seconds. In some embodiments, the actuation time is between about 30 seconds and 35 seconds, 30 seconds and 40 seconds, 30 seconds and 45 seconds, 30 seconds and 50 seconds. In some embodiments, the actuation time is between about 30 seconds and 35 seconds, 30 and 40 seconds, 30 seconds and 45 seconds, or 30 seconds and 50 seconds. In some embodiments, the actuation time is between about 35 and 40 seconds, 35 seconds and 45 seconds, or 35 seconds and 50 seconds. In some embodiments, the actuation time is between about 40 seconds and 45 seconds or 40 seconds and 50 seconds.

In some embodiments, the PEG having a molecular weight of 6,000 g/mol is used because it maintains stability (including remaining in solid state) during standard shipping conditions.

In some embodiments, as described herein, the wax is provided in the wax valve channel 110 via a wax fill port (see 144 in FIGS. 3A, 3B, 6, and 9). In some embodiments, the wax is provided in a molten state, which then solidifies within the wax valve channel 110. In some embodiments, the amount of the wax provided within the wax valve channel 110 is from about 1 uL to about 10 uL, from about 2 uL to about 8 uL, from about 3 uL to about 6 uL, or from about 4 uL to about 5 uL. In some embodiments, no specific patterning or configuration of the wax is needed within the wax valve channel 110.

In some embodiments, as described herein, the substrate includes a valve heater 135 configured to supply heat to the thermally-sealed valve for softening, melting, and/or dissolving thereof. In some embodiments, the wax valve channel 110 is spaced further apart from the substrate 109 with respect to elevation than for example, a bottom of the incubation chamber 112 that is aligned with the mixing heater 134. Accordingly, with reference to FIG. 6, in some embodiments, the assay device further comprises thermal conductive material 137 (e.g., a thermal pad) disposed between the substrate and the thermally-sealed valve (in the wax valve channel 110), so as to relay heat therebetween. For example, in some embodiments, the thermal conductive material comprises GAP PAD®, 3M thermally conductive adhesive transfer tape, ARTIC Thermal Pad, Laird Tflex, Fujipoly CARCON gap filler pad, or an equivalent material.

In some embodiments, as described herein, the substrate comprises a power supply to supply power to the valve heater 135 for heating thereof. In some embodiments, the power supply is the same as the power supply for the mixing heater 134. In some embodiments, the power supply is different from the power supply for the mixing heater 134. In some embodiments, the power supply is external to the substrate. In some embodiments, the valve heater is a resistive heater. The heater can either be regulated to control for a given temperature, or it can be heated at a constant power until fluid is detected in the reaction wells (which would indicate that the thermally sealed valve is open).

In some embodiments, as depicted in FIG. 6, one or more walls 148 of the entrance 146 to the wax valve channel 110 from the incubation chamber 112 is converging (e.g., sloped). In some embodiments, having converging walls at the entrance 146 of the wax valve channel 110 helps reduce or eliminates the formation of gas bubbles in the wax valve channel and/or along the valve-fluid interface (e.g., see 150 in FIG. 10). As described herein (see for example, Example 1), in some embodiments, the thermally-sealed valve is configured to dissolve (e.g., slowly dissolve) upon contact with certain liquids (e.g., the buffer with the sample solution). In some embodiments, hydrostatic pressure from the sample solution helps provide force to push through a softened, melted and/or dissolved valve forward through the wax valve channel 110, wherein shearing of the valve caused by movement against the wall of the wax valve channel 110 may further increase dissolution (of the valve). Accordingly, in some embodiments, such dissolution of the valve, by contact with the sample solution, along with hydrostatic pressure imposed by the sample solution within the wax valve channel to the thermally-sealed valve, helps decrease the pressure and time required to open said valve (e.g., via softening and/or melting), so as to allow the prepared sample solution to flow through the wax valve channel and to the OPM module 108. In some embodiments, such opening of the valve may occur at temperatures above or about the values known in the art for the wax melting point, or in some cases, at temperatures lower than values known in the art for the wax melting point.

Solubility of the thermally sealed valve material is also increased by temperature. Therefore actuation (opening) of the thermally sealed valve (which is the dissolution of the thermally sealed valve material) occurs faster when the temperature of the assay device or proximal to the wax valve channel is higher.

In some embodiments, the amount of time the thermally-sealed valve can prevent the sample solution from flowing through the wax valve channel 110 is a function of the sample solution temperature (e.g., within the incubation chamber 112), the valve actuation temperature, the cross-sectional area of the wax valve channel, and/or the length of the wax valve channel blocked by the valve. In some embodiments, the channel diameter is between about 100 μm and 2 mm. In some embodiments, the wax bolus within the channel is between about 100 μm and 10 mm—it can vary depending on the length of the wax valve channel.

In some embodiments, when the sample solution temperature approaches the valve melting temperature, the integrity of the valve may be compromised and thus unable to reliably prevent the sample solution from flowing through the wax valve channel 110. Accordingly, in some embodiments, the assay device includes a thermocouple detecting a temperature of the sample solution within the incubation chamber 112 during the incubation/mixing phase, such that if the temperature of the sample solution approaches a prescribed range of the valve melting temperature, the controller coupled to the mixing heater 134 is configured to stop supplying power to the heater. In some embodiments, the thermocouple is present and facilitates the prevention of the assay reaching a specific temperature or temperature range in the incubation chamber.

In some embodiments, the presence of a gas bubble (e.g., air bubble) results in the valve opening at a temperature higher than literature values, due to limited or lack of prior dissolution via contact with the sample solution, and/or due to limited or reduced hydrostatic pressure, thereby resulting in a reduced force received by the valve to be pushed open and across the wax valve channel 110.

In some embodiments, as described herein, the assay device is provided with the valve solidified therein. For example, in some embodiments, the assay device may be assembled and packaged with the valve therein, and thus only contacts a liquid upon use by a subject when performing an assay, so as to prevent potential slow and eventual dissolution of the valve prior to introducing the sample solution into the incubation chamber 112.

In some embodiments, once the prepared sample solution flows through the wax valve channel, additional unmixed sample solution may flow from the sample inlet and/or the sample preparation device 104 (which may remain coupled to the assay device 102 after the mixing/incubation phase). Accordingly, in some embodiments, the entrance 146 to the wax valve channel 110 is spaced as far apart from the sample inlet 118 so as to help reduce the risk and/or amount of unmixed (and thereby possibly non-lysed) sample from entering the OPM module.

### Optical Property Modifying Module

As described herein, in some embodiments, the sample solution, after being mixed and/or incubated for a sufficient period of time (thus a prepared sample solution), will flow through the wax valve channel 110 once the thermally-sealed valve is opened, and into the optical property modifying (OPM) module 108. In some embodiments, the OPM module 108 comprises a device as described in PCT/US2018/044044 (“the '044 Application”), which is incorporated herein in its entirety. In some embodiments, the common sample receiving inlet in the '044 Application is in fluidic communication with the wax valve channel 110, so as to receive the sample therefrom. In some embodiments, OPM module 108 comprises a reaction chamber channel 152 (FIGS. 2A and 2B) in fluidic communication with the wax valve channel 110 instead of or in addition to the common sample receiving inlet in the '044 Application. In some embodiments, the reaction chamber channel 152 continues along the OPM module 108 about a center portion 156, having one or more branches to a corresponding reaction chamber 154. In some embodiments, the first and second piece as described in the '044 Application are each part of a top and bottom portion of the assay device 102 as described herein. In some embodiments, the OPM module 108 is a separable device couplable to the wax valve channel 110 and thermal mixing module 106.

In some embodiments, the wax is dissolved as the prepared sample solution enters the reaction chamber channel 152 in the OPM module 108. In some embodiments, the prepared sample solution is distributed to the one or more reaction chambers 154 via the one or more branches off the reaction chamber channel 152.

In some embodiments, the OPM module 108 further comprises a sequestration chamber 158 which is coupled to a first branch off the reaction chamber channel 152. See FIG. 2A. In some embodiments, the sequestration chamber is vented. As described herein in Example 2, in some cases, a high amount of the dissolved valve material will be found in the initial volume of the prepared sample solution entering the OPM module 108, and thus the sequestration chamber 158 will obtain a large amount (e.g., excess) of the dissolved valve material, reducing the amount of the dissolved valve material distributed across the one or more reaction chambers 154.

In some embodiments, the OPM module 108 comprises a series of one or more mixing chambers (e.g., shuttle mixing chambers) configured to help distribute the dissolved thermally sealed valve material throughout the reaction chambers more uniformly. In some embodiments, such one or more shuttle mixing chambers are arranged in series connected to each other using a narrow shuttle mixing channel, wherein at least one shuttle mixing chamber is configured to contain the entire sample solution volume. In some embodiments, the one or more shuttling mixing chambers are disposed upstream of the reaction chambers.

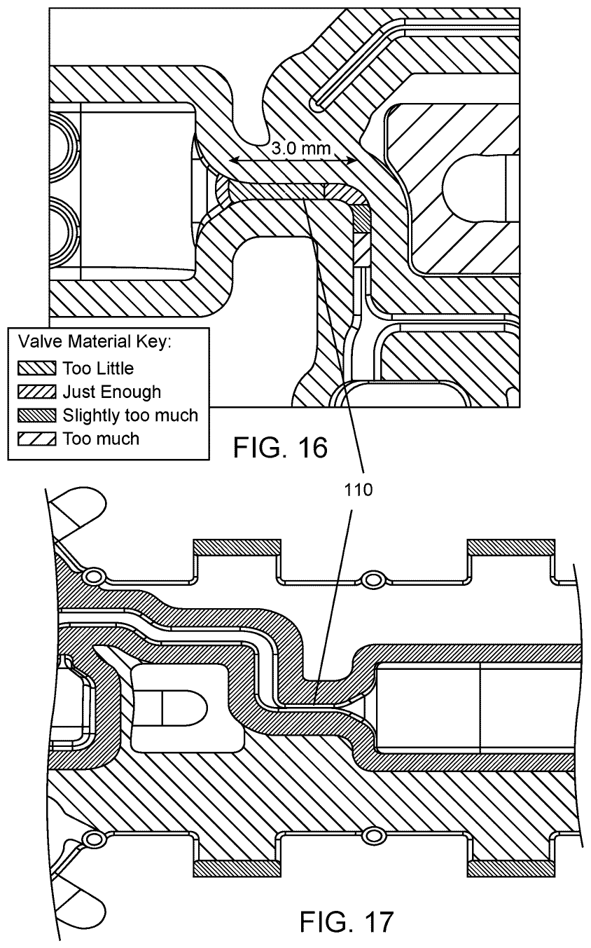

In some embodiments, the wax valve channel tapers proximal to the wax fill port. In some embodiments, the wax fill port is narrow (e.g., about 500 um) proximal to the wax fill port and widens distal to the wax fill port (e.g., 1 mm at the distal end). In some embodiments, the ratio of the narrowest end of the wax valve channel to the widest end of the wax valve channel is about 1:2 or lower (e.g. 1:2.5, 1.3, etc.). This wax valve channel geometry allows the thermally sealed valve to maintain a consistent length when dispensed within the channel (i.e., the same length or less than the heated area with varying volumes of dispensed thermally sealed valve material-any excess thermally sealed valve material is dispensed into the wider sections of the channel without the overall length of the valve increasing significantly. This can also minimize excess thermally sealed valve material entering the OPM. See, e.g., FIG. 16 showing a wax valve channel geometry that does not taper proximal to the wax fill port or widen on the distal to the wax fill port and can allow entry of excess thermally sealed valve material in the OPM. Alternatively, FIG. 17 shows a wax valve channel 110 that is narrow proximal to the wax fill port and widens on the end distal to the wax fill port.

### Sample Preparation Devices

In some embodiments, the sample preparation device comprises any sample preparation device as described in any of U.S. Pat. Nos. 11,123,736 and 11,125,661, the disclosures of which are incorporated herein in their entireties.

### Method for Preparing Thermally Sealed Valve

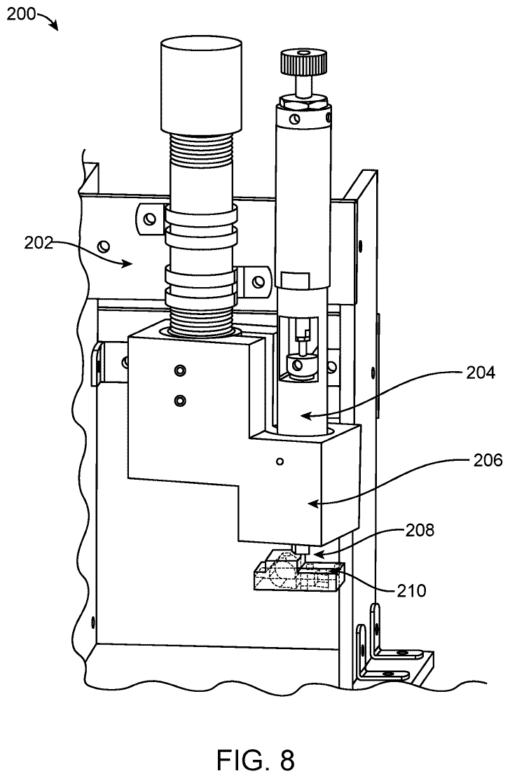

With reference to FIG. 9, a method for preparing a thermally sealed valve within a wax valve channel 110 of an assay device is depicted. In some embodiments, an assay device is provided, as described herein, having a thermal mixing module, a wax valve channel 110, and a OPM module (or optionally, the OPM module is removably couplable to the wax valve channel 110). In some embodiments, the wax valve channel 110 includes a valve fill port 144 that allows for the thermally sealed valve to be inserted into the wax valve channel 110. In some embodiments, the thermally sealed valve material is dispensed into the wax valve channel 110 in a molten or dissolved state. FIG. 8 depicts an exemplary device 200 for dispensing the thermally sealed valve material having a pressurized wax reservoir 202, a precision dispensing valve 204, a heater block 206, and/or a wax dispenser needle 208. In some embodiments, the wax dispensing device 200 is a pneumatic-driven system. Using the device 200, a thermally sealed valve material can be dispensed onto an integrated test chamber 210. Many other systems in the art may require heating the plastic device where the thermally sealed valve material is deposited. In the devices of the present disclosure, the assay device does not have to be heated in order to dispense the thermally sealed valve material. In some embodiments, the wax dispenser device and/or consumable are configured to dispense from about 1 uL to about 10 uL, about 2 uL to about 7 uL, about 3 uL to about 5 uL, or about 4 uL of thermally sealed valve material into the wax valve channel 110.

In some embodiments, once a sufficient amount of thermally sealed valve material has been dispensed in the wax valve channel 110, the valve fill port 144 is sealed. In some embodiments, extra polymer is melted around the fill port 144, which the solidifies after cooling and thereby sealing the wax fill port 144 opening. In some embodiments, a stopper is introduced to the wax fill port 144 opening. In some embodiments, a pressure-sensitive adhesive is placed over the wax fill port 144 opening to seal thereof. In some embodiments, the wax fill port opening is left opened. In some embodiments, the plastic of the wax fill port is melted and heat staked closed. For example, a material such as the device plastic is melted over the wax fill port to seal thereof.

In some embodiments, the molten or dissolved thermally sealed valve material (also referred to as dissolved valve material) will flow into the wax valve channel 110 and solidify accordingly, without need to shape and/or position the thermally sealed valve material within the wax valve channel 110. In some embodiments, the thermally sealed valve material and/or wax valve channel is dried and/or cooled prior to packaging, so as to ensure solidification of the thermally sealed valve material within the wax valve channel 110.

### Method for Performing a Biological Assay in System

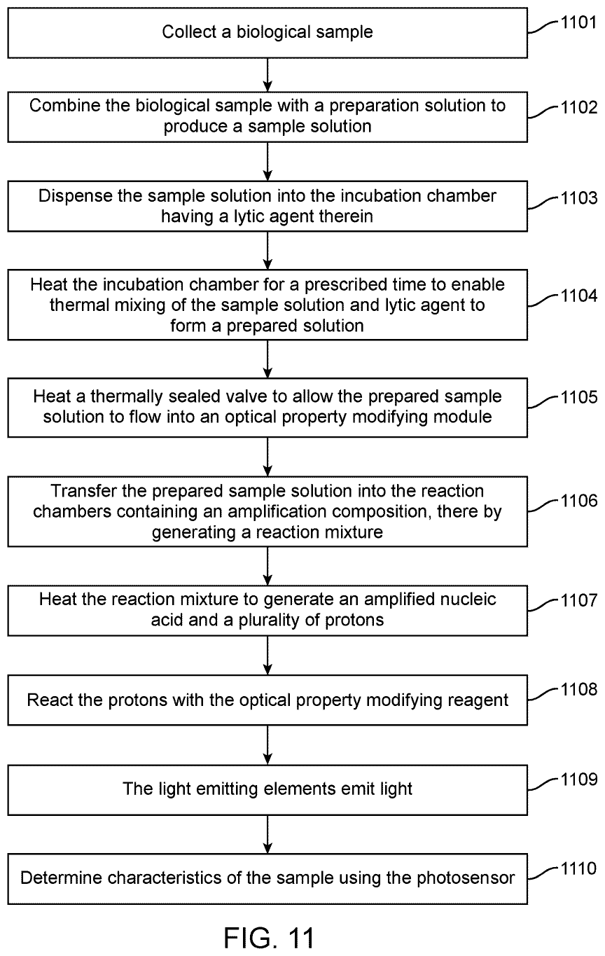

FIG. 11 is a flow chart of a method 1100 for performing a biological assay using a system (e.g., 100), in accordance with an embodiment described herein. In other embodiments, the method can include different and/or additional steps than those shown in FIG. 11. Additionally, steps of the method can be performed in different orders than the order described in conjunction with FIG. 11 in various embodiments.

A subject provides 1101 a biological sample. As described herein, the biological sample is a sample containing a quantity of organic material, e.g., one or more organic molecules, such as one or more nucleic acids e.g., DNA and/or RNA or portions thereof, which can be taken from the subject. In some aspects, the biological sample is a nucleic acid amplification sample, which is a sample suspected of including one or more nucleic acids or portions thereof which can be amplified.

The provided biological sample can include one or more cells, such as tissue cells of the subject. As used herein, the term “tissue” refers to one or more aggregates of cells in a subject (e.g., a living organism, such as a mammal, such as a human) that have a similar function and structure or to a plurality of different types of such aggregates. Tissue can include, for example, organ tissue, muscle tissue (e.g., cardiac muscle; smooth muscle; and/or skeletal muscle), connective tissue, nervous tissue and/or epithelial tissue. Tissue can, in some versions, include cells from the inside of the subject's cheek and/or cells in the subject's saliva.

The provided liquid sample (e.g., biological sample) can include, for example, human saliva, urine, human mucus, blood, an oral rinse, or a solid tissue such as buccal tissue. The biological sample can also include bacteria or spores. The biological sample can be provided by a sample collector. Providing can include contacting, e.g., rubbing and/or scraping, the sample collector against one or more surfaces of the subject and/or surfaces of the biological sample of the subject, such as a liquid, e.g., saliva and/or blood, sample extracted from the subject. As such, in some versions, providing includes extracting one or more biological samples from the subject. In some versions, providing the biological sample can include instructing the subject to produce the biological sample, such as by spitting onto and/or into the sample collector. Providing the biological sample can also include retaining the biological sample or a portion thereof, e.g., one or more cells, on the sample collector while, for example transferring the sample collector to a sample preparation device (e.g., 104) or an assay device (e.g., 102). In some instances, the sample collector is a swab and providing the biological sample includes swabbing the inside of the subject's mouth and/or nose to obtain the biological sample on the collector. In some versions, sample collectors are nasopharyngeal, mid-turbinate, genital, and/or nasal swabs. After the biological sample is provided, the method 1100 can include mixing the biological sample with a preparation solution to form a sample solution 1102. In some embodiments, the biological sample is mixed with a preparation solution in a sample preparation device (e.g., 104) as described herein.

In some embodiments, the preparation solution comprises an optical property modifying reagent solution. In some embodiments, the optical property modifying reagent solution comprises an optical property modifying reagent and a liquid buffer.

Optical property modifying reagents can include, for example, pH sensitive dyes, fluorescent dyes, FRET dyes, micro and nano particles, fluorescent proteins, colorimetric substrates, enzymes and reagents, plasmonic structures, precipitation reagents and substrates, or any combination thereof.

In some versions, the optical property modifying reagent is or includes an enzyme-linked immunosorbent assay (ELISA) reagent. In some aspects, the ELISA reagent is selected from the group consisting of alkaline phosphatase, horseradish peroxidase, β-galactosidase, BCIP/NBT (5-bromo-4-chloro-3-indolyl-phosphate/nitrobluetetrazolium), TMB (3,3′,5,5′ tetramethylbenzidine), DAB (3,3′,4,4′ diaminobenzidine), 4CN (4-chloro-1-naphthol). TMB (dual function substrate), ABTS (2,2′-azino-di [3-ethylbenzthiazoline] sulfonate), OPD (o-phenylenediamine), MUG (4-methylumbelliferyl galactoside), HPA (hydroxyphenylacetic acid), and HPPA (3-p-hydroxyphenylproprionic acid).

Optical property modifying reagents, in various instances, can include one or more optical property modifying substances and as such, be configured to have one of their optical properties, such as color, modified. As such, the method 1100 includes modifying one or more optical properties of an optical property modifying reagent.

Modifying an optical property refers to changing one or more optically-recognizable characteristics of an aspect, e.g., a sample, such as a characteristic resulting from wavelength and/or frequency of radiation, e.g., light, emitted from an aspect, such as color, fluorescence, phosphorescence, etc. For example, in some versions, the optical property is color and modifying the optical property includes changing the color. In some aspects, such an optical property modification, e.g., color change, is detectable by an unassisted human eye under, for example ambient light. In alternative aspects, such as the method 1100, the optical property modification is detectable using a photosensor. Modifying an optical property can also include changing the transmittance and/or opacity of a substance and can include causing the substance to change substantially from transparent to opaque or from opaque to transparent. As such, the method 1100 can include detecting such a change in transmittance with a photosensor.

The user dispenses 1103 the sample solution into the assay device, or specifically the incubation chamber (e.g., 112) of the thermal mixing module via a sample receiving module (e.g., 114) (see for example FIG. 10). In some embodiments, the incubation chamber 112 has a lytic agent (e.g., 136) therein for mixing with the sample solution so as to form a prepared sample solution. Such mixing helps prepare the biological sample within the sample solution to react, for example, with assay reagents and/or an optical property modifying reagent upon exposure thereto. The mixing can include lysing cells of the biological sample with the lytic agent and/or extracting nucleic acids therefrom. Such extracted nucleic acids can be released into the resulting prepared sample solution. In some embodiments, a step of extracting genomic deoxyribonucleic acid (DNA) from the biological sample is included. In some embodiments, the lytic agent is provided as a pellet that may be lyophilized, and which is hydrated once exposed to the sample solution.

The incubation chamber is then heated 1104 using a mixing heater 134 disposed below thereof, so as to enable thermal mixing of the sample solution with the lytic agent. In some embodiments, the mixing heater 134 is aligned to be off-set from a center portion of the incubation chamber 112 to help promote uneven buoyancy of the sample solution, thereby promoting fluid movement within the chamber 112 (for e.g., the portion of the sample solution within the vicinity of the mixing heater will become higher in temperature, thus lowering in density, and allow to rise and move within the chamber). In some embodiments, the incubation chamber comprises one or more rounded walls 138 to help facilitate a circulating, and in some cases, a circular or somewhat similar motion within the incubation chamber 112. In some embodiments, the mixing heater 134 provides heat based on receiving power from a power supply at a constant rate, or by having the power regulated such that the mixing heater temperature 134 remains constant or substantially constant. In some embodiments, the sample solution is mixed and/or incubated for a prescribed time. In some embodiments, the prescribed time is from about 1 minute to about 20 minutes.

After the sample solution has been mixed for the prescribed time, a valve heater 135 is then used to apply heat to a thermally sealed valve 1105 (see for example, FIG. 10). In some embodiments, the thermally sealed valve is located within a wax valve channel 110 that extends from an end of the incubation chamber 110 opposite from an inlet of the sample receiving module 114. In some embodiments, the thermally sealed valve provides a barrier to prevent the sample solution from passing through the wax valve channel 110. In some embodiments, the thermally sealed valve is a solid at a first temperature, but is configured to soften and/or melt once exposed to sufficient heat (e.g., the valve heater 135). In some embodiments, the thermally sealed valve comprises a wax or a water-soluble polymer (e.g., PEG). In some embodiments, the valve heater heats the thermally sealed valve, which in some cases, has a melting temperature from about 40° C. to about 65° C. In some embodiments, the thermally sealed valve softens, melts and/or dissolves sufficiently to be opened by the prepared sample solution, and thereby allows the prepared sample solution to flow through the wax valve channel 110 to an optical property modifying (OPM) module 108. In some embodiments, the thermally sealed valve dissolves into the prepared sample solution.

The prepared sample solution enters a reaction chamber channel 152 in the OPM module 108 that is in fluidic communication with one or more reaction chambers 154. The prepared sample solution is then transferred into the reaction chambers 154 (step 1106). Specifically, the prepared sample solution travels along the reaction chamber channel 152 and branches off into one or more reaction chambers 154, wherein the reaction chambers 154 comprise assay reagents, thereby generating a nucleic acid reaction mixture.

As described herein, in some embodiments, each of the reaction chambers 154 comprises assay reagents. As such, transferring the prepared sample solution into one or more of the reaction chambers can include mixing the prepared sample solution with the assay reagents, and thereby generating the nucleic acid reaction mixture including the prepared sample solution and the assay reagents for carrying out a nucleic acid amplification reaction.

The assay reagents comprise enzymes and nucleic acid primers capable of reacting with a biological sample such that one or more nucleic acids suspected to be present within the sample can be amplified, if present, e.g., amplified isothermally. In certain embodiments, the assay reagents comprises nucleic acid amplification enzymes and DNA primers. For example, the assay reagent can include one or more primers, deoxynucleotides (dNTPs), and/or polymerases, Trizma pre-set crystals (Tris buffer, pH 8.8; Sigma, cat. no. T9443), Potassium chloride (KC1; Wako Pure Chemicals, cat. no. 163-03545), Magnesium sulfate heptahydrate (MgS04; Wako Pure Chemicals, cat. no. 137-00402), Ammonium sulfate (( H4)2S04; Kanto Chemical, cat. no. 01322-00), Tween 20 (Tokyo Chemical Industry, cat. no. T0543), Betaine solution (Betaine, 5 M; Sigma, cat. no. B0400), Calcein (DOJINDO, cat. no. 340-00433) plus one or more optical modification reagents as discussed above, Manganese(II) chloride tetrahydrate (MnCl2; Wako Pure Chemicals, cat. no. 133-00820), Agarose S, EtBr solution, template nucleic acids, or any combination thereof. In addition, in some versions, the assay reagents, can be stored in the fluidic chambers 205 in dry, e.g., lyophilized, form. As such, preparing the reaction mixture can include mixing the prepared sample solution and the assay reagents and/or hydrating the assay reagent.

The assay reagents can comprise one or more reagents capable of amplifying nucleic acids present in a biological sample via an isothermal amplification protocol including: transcription mediated amplification, strand displacement amplification, nucleic acid sequence-based amplification, rolling circle amplification, loop-mediated isothermal amplification, isothermal multiple displacement amplification, helicase-dependent amplification, circular helicase-dependent amplification, single primer isothermal amplification, loop-mediated amplification, or any combination thereof. In certain embodiments, the amplification reaction performed is LAMP. In a LAMP reaction, a double- or single-stranded DNA template in dynamic equilibrium at an elevated temperature is amplified using two or three pairs of primers. The primers are designed based on the DNA template, using primer design software such as LAMP Designer (Premier Biosoft, Palo Alto, CA). In the first step of the LAMP reaction, the F2 region of the FIP (Forward Inner Primer) anneals to the single stranded DNA at the respective complementary (F2c) position. Next, a polymerase with strand displacement activity incorporates dNTPs along the template from the 3′ end of F2. The incorporation of nucleotides releases protons, reducing the pH of the reaction mix. Then, the F3 forward primer anneals to the F3c region upstream of the F2 region and on the template. The F3 forward primer begins amplifying the template strand, which releases further protons and displaces the FIP-incorporated strand that was synthesized previously. This single strand contains an F1 sequence (within the target sequence) along with its complementary F1c sequence (within the FIP). This forms a stem-loop as F1c anneals to F1 at the 5′ end. At the same time, the BIP (Backward Inner Primer) anneals to the other end of the strand and nucleotides extend from B2, releasing more protons. The backward primer B3 then binds to the B3c region, downstream of the B2 region, displaces the BIP-amplified strands and promotes extension to create the double strand. This displaced strand now contains a B1 sequence (within the target sequence) along with its complementary B1c sequence (within the BIP), forming another stem loop in the 3′ end. The structure now has two stem-loop structures at each end from which continuous displacement and extension occur to amplify the template. The LAMP reaction can be amplified by adding further Forward and Backward Loop primers to produce more amplicons with stem loop structures.

The LAMP procedure can take place at a fixed temperature, minimizing the need for any expensive thermocycling equipment. Typically, isothermal methods require a set temperature, which is determined by the selected reagents. For example, enzymes function best between 60-65° C. in LAMP methods. Amplification according to the subject embodiments can also be performed by applying PCR.

In some embodiments, the system (e.g., 100) heats 1107 the reaction mixture (using for example, a reaction heater) to generate an amplified nucleic acid and a plurality of protons. Specifically, heating the reaction mixture with a reaction heater promotes a nucleic acid amplification reaction using the nucleic acid from the biological sample and the assay reagents. This reaction generates the amplified nucleic acid and the plurality of protons.

In some embodiments, the heating step 1107 includes transferring thermal energy from the reaction heater to a thermal gap pad to one or more of the reaction chambers. Heating the reaction mixture promotes the nucleic acid amplification reaction between, the nucleic acids of the biological sample and the assay reagent. This nucleic acid amplification reaction generates the amplified nucleic acid and the plurality of protons.

The protons then react 1108 with the optical property modifying reagent. Reacting the reaction product, or an aspect thereof, with an optical property modifying reagent can include chemically modifying the reaction product and/or the optical property modifying reagent, such as by bonding the one or more protons to the optical property modifying reagent. In some embodiments, this reacting of the protons with the optical property modifying reagent sufficiently modifies an optical property of the optical property modifying reagent to allow detection of the modified optical property indicative of the presence of a suspected analyte in the biological sample.

The assay device causes 1109 light emitting elements to emit light. Specifically, a microprocessor of the system (as described in the '044 Application) instructs the plurality of light emitting elements to emit light in a repeating pattern at a repetition frequency. During the repeating pattern, each light emitting element of the plurality of light emitting elements emits light at a distinct time point such that only one of the plurality of reaction chambers is illuminated at any time. Exposure to light can provide a change in conditions such that optical properties can be measured. In this way, during each repeating pattern, optical properties of the contents of each reaction chamber can be continuously monitored by a photosensor.

Based on the optical properties detected in step 1109, the system (e.g., 100) is able to determine 1110 one or more characteristics of the samples contained in the reaction chambers using a photosensor and a microprocessor, which performs an optical property analysis of the reaction mixtures in the one or more the reaction chambers, as described in the '044 Application. Performing the optical property analysis can include determining whether a change in an optical property of one or more contents of the reaction chambers has occurred.

Optical property analysis can be performed in real-time throughout the amplification reaction described with regard to step 1106, or after the performance of the amplification reaction. Detection of the modified optical properties of the reaction mixture can be associated with a digital indication of a presence or absence of the amplification reaction product. In other words, detection of the modified optical property of the reaction mixture can provide information regarding whether the amplification reaction product is present or absent. In certain embodiments, detection of a modified optical property of the reaction mixture indicates that the exponential or plateau phase of the amplification reaction has been obtained.

In some embodiments, detection of the amplification reaction product is accelerated relative to an amplification reaction that uses a reaction mixture without a halochromic agent. In further embodiments, the optical property modification of the reaction mixture is detected in less than 60 minutes from a starting time of the amplification reaction. Accelerated detection of the amplification reaction product is obtained because the halochromic agent (a weak acid or base) in the reaction mixture absorbs protons generated during the amplification reaction, and recombination of the free protons acts to accelerate the detection of the amplification reaction. The reaction can be designed so that minimal amplification is required to generate a pH transition sufficient for the halochromic agent to change optical property. Conventional amplification techniques that use fluorescent intercalating dyes, molecular beacons, hybridization probes, dye-based detection, UV-Vis, or other detection methods require a certain threshold amount of amplification to occur before an amplification signal is detectable. However, the methods of the present disclosure require a relatively smaller threshold amount of amplification before an optical property modification of the halochromic agent is detectable, and therefore the detection of an amplification reaction product is accelerated relative to conventional amplification methods.

In some embodiments, the system is configured to display the determined characteristics using an electronic result display mechanism (as described in the '044 Application). The results provided can be in the form of a visual output on a display and/or in the form of an audio output.

Of note with regard to the method 1100 is that in various embodiments, the system comprises one or more, e.g., three, assay controls: a sample adequacy control, a positive control, e.g., an internal positive control, and/or a negative control. The sample adequacy control detects, for example, abundant human nucleic acid markers such as housekeeping genes, RNA, and/or human ß-actin deoxyribonucleic acid (DNA) to ensure a sufficient swab sample was provided. The positive control amplifies a synthetic oligonucleotide that can be co-packaged and/or co-lyophilized within the reaction chambers. Such a synthetic oligonucleotide can be included, for example, in the optical property modifying reagent solution and/or in the assay reagents. Such a control ensures that the system operates under conditions that allow amplification of genetic markers of interest. The negative control also amplifies the positive control but without the co-lyophilized synthetic oligonucleotide. Such a control ensures the absence of any contaminating self-amplifying amplicon. KITS

The embodiments disclosed herein also include kits including the subject devices and which can be used according to the subject methods. The subject kits can include two or more, e.g., a plurality, three or less, four or less, five or less, ten or less, or fifteen or less, or fifteen or more, assay devices or components thereof, according to any of the embodiments described herein, or any combinations thereof.

The kits can include one or more compositions and/or reagents, such as any of those described herein, e.g., optical property modifying reagents, amplification compositions, preparation solutions and/or buffers, which can be stored in the kits in containers separate from the devices. In addition, the kits can include any device or other element which can facilitate the operation of any aspect of the kits. For example, a kit can further include one or more devices for preparing a sample and/or analyzing one or more characteristics of a sample, e.g., a prepared sample. In some embodiments, the kit comprises a sample preparation tube with one or more compositions and/or reagents. Kits can also include packaging, e.g., packaging for shipping the devices without breaking.

In certain embodiments, the kits which are disclosed herein include instructions, such as instructions for using devices. The instructions for using devices are, in some aspects, recorded on a suitable recording medium. For example, the instructions can be printed on a substrate, such as paper or plastic, etc. As such, the instructions can be present in the kits as a package insert, in the labeling of the container of the kit or components thereof (i.e., associated with the packaging or sub-packaging etc.). In other embodiments, the instructions are present as an electronic storage data file present on a suitable computer readable storage medium, e.g., Portable Flash drive, CD-ROM, diskette, on the cloud, etc. The instructions may be storable and/or reproducible within one or more programs, such as computer applications. In some embodiments, the instructions are accessible by scanning a QR code or bar code printed on a device component, a substrate, such as paper or plastic, etc. The instructions can take any form, including complete instructions for how to use the devices or as a website address with which instructions posted on the world wide web can be accessed.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T .E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1—Distribution of Dissolved Valve Material Across Reaction Chambers

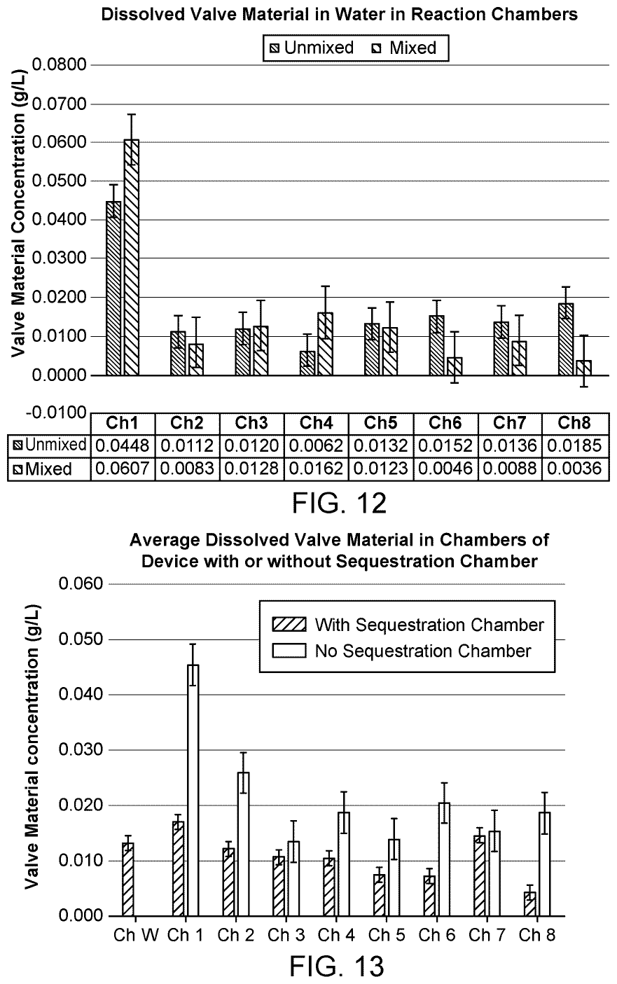

FIG. 12 depicts a distribution of thermally sealed valve material (e.g., polyethylene glycol) dissolved in a sample solution across the plurality of reaction chambers (Ch 1 to Ch 8) in an assay device. Two cases were shown, with a first case indicating a sample solution (water) where thermal mixing (as described herein) did not occur (unmixed group), while a second case indicates a sample solution (water) after thermal mixing (mixed group). In both cases, it is clear that the largest concentration of the dissolved thermally sealed valve material is found in the first chamber, while the remaining chambers have a relatively uniform and significantly smaller volume of thermally sealed valve material. This highlights a technical advantage of having a sequestration chamber (collection chamber for excess dissolved thermally sealed valve material, e.g., FIG. 2A) upstream of the reaction chamber(s), so as to remove a substantial amount of the dissolved valve material (e.g., the majority) prior to distributing the prepared sample solution in the reaction chamber(s). This also has the advantage of minimizing variation in the assay results from the different reaction chambers, since a sequestration chamber allows for a relatively uniform distribution of dissolved valve material in each of the chambers.

FIG. 13 includes a plot showing the distributions of dissolved valve material in reaction chambers in an assay device having a sequestration chamber (e.g., 158 in FIG. 2A) and an assay device having no sequestration chamber (e.g., FIG. 2B). Ch W represents the sequestration chamber. Ch 1 to Ch 8 represents the reaction chambers. The results showed dissolved valve material was present in all the reaction chambers whether or not a sequestration chamber was used, and in the case of no sequestration chamber, the distribution of the dissolved valve material was comparable across Ch 2 to Ch 8. In the assay device group having no sequestration chamber, there was a disproportionate but still small volume of dissolved valve material present in the first reaction chamber (Ch 1).

### Example 2—Impact of Liquid Dissolution and Hydrostatic Pressure on Actuation Temperature of Thermally Sealed Valve Material

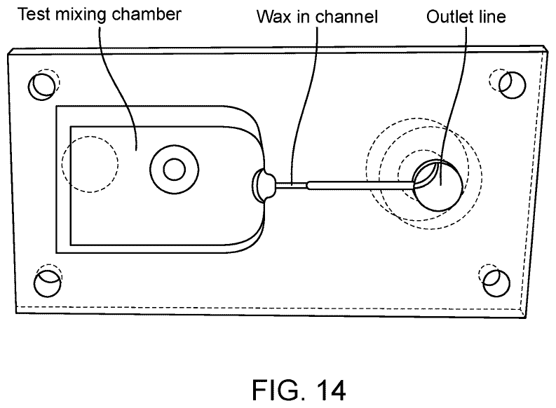

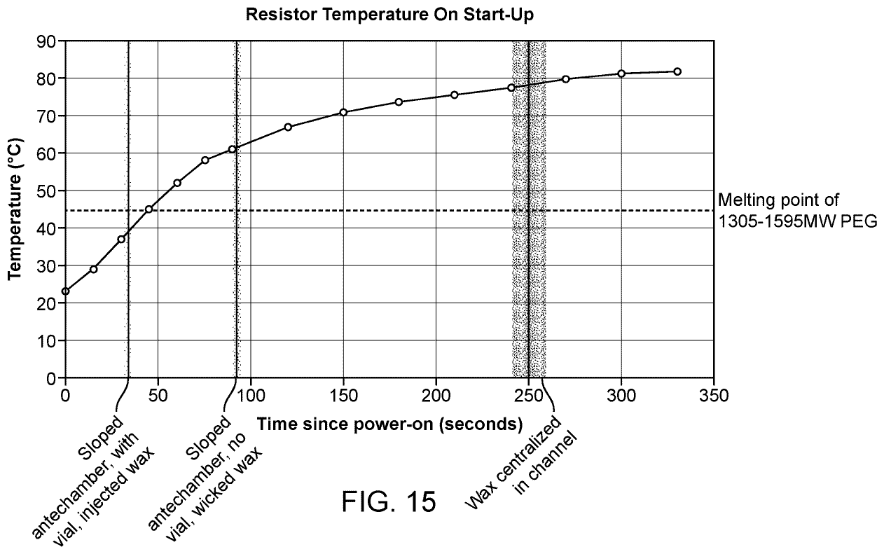

A testing apparatus having some of the assay elements described herein (e.g., mixing chamber and wax valve channel), as illustrated in in FIG. 14, was used to test actuation temperature for thermally sealed valve material. FIG. 15 includes a plot depicting the actuation temperature of a thermally sealed valve (e.g., PEG) under differing assembly conditions. The literature value of PEG having a molecular weight from 1305 to 1595 is about 44° C. to about 46° C. In a first condition, a wax valve channel on an assay device was provided without a sloped entrance from an incubation chamber, wherein the thermally sealed valve was centralized in the channel, had air in between the fluid (e.g., liquid, such as a buffer) and the thermally sealed valve material, such that there was reduced or no dissolution by the liquid on the thermally sealed valve material, and limited or no hydrostatic pressure imposed by the liquid. The resulting actuation temperature was 77° C.-79° C.

In a second condition, a sloped entry to the wax valve channel was provided, wherein a liquid was in contact with the thermally sealed valve, but limited or no hydrostatic pressure was exerted by the liquid on the thermally sealed valve. Accordingly, the resulting actuation temperature was 61° C.-63° C., thus indicating the dissolution of the thermally sealed valve by exposure to liquid reduced the actuation temperature by 16° C. as compared to having no contact with liquid.

In a third condition, a sloped entry to the wax valve channel was provided, along with a vertically oriented vial providing a liquid, such that a hydrostatic pressure was imposed by the liquid onto the thermally sealed valve. Accordingly, the resulting actuation temperature was 38° C.-39° C., thus indicating the dissolution of the thermally sealed valve by exposure to liquid along with hydrostatic pressure further reduced the actuation temperature, in this case below the literature value.

Table 1 provides the injection parameters for a PEG-based thermally sealed valve in an assembly of the present disclosure. The PEG used had a molecular weight of 6,000 g/mol.

TABLE 1
Injection Parameters for PEG 6000MW.

| Temperature of | Dispense | Back | | |
|---|---|---|---|---|
| Thermally Sealed | Actuator | Pressure on | Dispense | Dispense |
| Valve Material in | Timing | Wax Reservoir | Vacuum | Pressure |
| Dispenser (° C.) | (sec) | (psi) | (psi) | (psi) |
| 85 | 0.060 | 1 | 0 | 70 |

The actuation time for the 6000 g/mol PEG was measured and averaged between 30 seconds and 50 seconds. See Table 2.

TABLE 2
Actuation times for the loaded PEG 6000MW.

| | Run 1 | Run 2 | Run 3 | Average |
|---|---|---|---|---|
| Duty Cycle | (mm:ss) | (mm:ss) | (mm:ss) | (mm:ss) |
| 25% | 0:49 | 0:50 | 0:50 | 0:49 |
| 37.50% | 0:35 | 0:44 | 0:46 | 0:41 |
| 50% | 0:32 | 0:26 | 0:45 | 0:34 |
| 67.50% | 0:28 | 0:28 | 0:39 | 0:31 |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims.

1. A system for performing a biological assay, the system comprising:
a. a thermal mixing module comprising:
i. a sample receiving module for receiving a sample solution comprising a biological sample and a preparation solution; and
ii. an incubation chamber in fluidic communication with the sample receiving module;
b. a wax valve channel in fluidic communication with the incubation chamber;
c. an optical property modifying (OPM) module operatively coupled with the incubation chamber via the wax valve channel, the OPM module comprising one or more reaction chambers each comprising an assay reagent;
d. a thermally sealed valve disposed (i) within the wax valve channel, and (ii) between the sample receiving module and the OPM module; and
e. a mixing heater configured to supply heat to the incubation chamber.

2. The system according to claim 1, wherein the incubation chamber comprises a lytic agent, the lytic agent comprising one of Dithiothreitol (DTT), Proteinase K, Mutanolysin, Lysostaphin, Lysozyme, a combination thereof, a lyophilized pellet, one or more surfactants, or one or more components of a buffer solution, and wherein the mixing heater is configured to heat the sample solution within the incubation chamber, thereby enabling mixing of the sample solution and the lytic agent therein to form a prepared sample solution, for a prescribed amount of time.

3. The system according to claim 1, wherein the mixing heater is configured to be aligned with at least a portion of the thermal mixing module and offset from a center portion of the incubation chamber.

4. The system according to claim 1, further comprising a sample preparation device configured to mate with the sample receiving module, and wherein the sample receiving module comprises:
a puncturing element configured to pierce a breakable seal on the sample preparation device, thereby enabling fluidic communication between a sample preparation chamber within the sample preparation device and the incubation chamber; and
a luer for coupling the sample preparation device to the assay device, wherein the sample preparation device comprises a collar that contacts the luer so as to form a leak tight seal between the sample preparation device and the sample receiving module when mated together.

5-8. (canceled)

9. The system according to claims 1, wherein the incubation chamber comprises a vent, wherein the vent is a selective venting elements comprising one of a self-sealing porous polymer vent plug, a self-sealing porous polyethylene vent, polytetrafluoroethylene, polyethersufone, or a hydrophobic membrane, and wherein the vent comprises a sensing channel in fluidic communication with a fill detection chamber configured to detect liquid filling thereof.

10-18. (canceled)

19. The system according to claim 1, wherein:
the thermal mixing module further comprises a light source and one or more sensors configured to (i) detect an initial presence of liquid within the incubation chamber, (ii) detect a liquid level in the incubation chamber, or (iii) both;
the one or more sensors are configured relative to the light source such that the one or more sensors detect light emitted by the light source and as the incubation chamber fills with liquid, the light detected by the sensor is attenuated;
a sensor of the one or more sensors is disposed within a fill-detection chamber in fluidic communication with the incubation chamber via a sensing channel, wherein said sensor is configured to detect a change in light within the fill-detection chamber; and
the one or more sensors are in operative communication with the mixing heater and serve as an interlock for the mixing heater, such that the mixing heater is configured to be activated and/or deactivated based on detection of a liquid and/or a liquid level within the incubation chamber by the one or more sensors.

20-52. (canceled)

53. The system according to claims 1, wherein the thermally sealed valve is solid or substantially solid at a first temperature, so as to help prevent the sample solution from flowing through the wax valve channel, and is configured to transition from the solid or substantially solid configuration to a soft, dissolved, and/or melted configured after receiving sufficient heat.

54. (canceled)

55. The system according to claims 1, further comprising:
a valve heater configured to heat the thermally sealed valve, thereby enabling the thermally sealed valved to be softened, dissolved, and/or melted to allow the sample solution to flow therethrough; and
one or more thermal conductive pads is operatively coupled with the mixing heater, the valve heater, or both, and configured to transfer heat from the mixing heater and/or the valve heater to the wav value channel so as to heat the thermally sealed valve.

56-60. (canceled)

61. The system according to claims 1, wherein the thermally sealed valve is configured to dissolve into the sample solution, the system further comprising a sequestration chamber located downstream from the was valve channel and upstream from the one or more reaction chambers, wherein the sequestration chamber is configured to receive the initial flow of the prepared sample solution and dissolved thermally sealed valve therein, so as to reduce the amount of the dissolved thermally sealed valve found in the one or more reaction chambers.

62. The system according to claim 1, wherein the prepared sample solution is configured to enter at least one reaction chamber of the one or more reaction chambers after passing through the wax valve channel.

63-65. (canceled)

66. The system according to claim 1, further comprising a substrate operatively coupled to the thermal mixing module, the wax valve channel, and/or the OPM module, wherein the substrate comprises at least one of a printed circuit board or a thermal gap pad, and wherein the substrate further comprises:
a power source operatively connected to the mixing heater and/or the valve heater. wherein the power source is configured to supply power to the mixing heater and/or the valve heater at a substantially constant rate; and
a controlled to regulate power supplied to the mixing heater and/or the valve heater so as to maintain the mixing heater and/or the value heater substantially at a predetermined temperature.

67-72. (canceled)

73. The system according to claim 1, wherein the preparation solution comprises a nucleic acid amplification preparation solution and an optical property modifying reagent.

74-75. (canceled)

76. The system according to claim 1, wherein the OPM module comprises:
a reaction chamber channel in fluidic communication with the wax valve channel, wherein the one or more reaction chambers are in fluidic communication with the reaction chamber channel through a corresponding branch;
a plurality of light pipes, each light pipe capable of transmitting light between one of the one or more reaction chambers and a single sensing region disposed in the OPM module; and
a reaction heater configured to heat the one or more reaction chambers,
wherein each reaction chamber is substantially equidistant from the single sensing region.

77-81. (canceled)

82. A method for determining one or more characteristics of a nucleic acid amplification sample based on a modified optical property of a biological sample, the method comprising:
b. providing the biological sample comprising a nucleic acid;
c. combining the biological sample with a preparation solution comprising a buffer solution and/or an optical property modifying reagent solution, so as to produce a sample solution;
d. dispensing the sample solution into an incubation chamber;
e. mixing the sample solution with a lytic agent using a mixing heater to apply heat to the incubation chamber, so as to enable thermal mixing, thereby forming a prepared sample solution;
f. heating a thermally sealed valve disposed within a wax valve channel in fluidic communication with the incubation chamber, so as enable the prepared sample solution to flow through the wax valve channel to one or more reaction chambers comprising an assay reagent, wherein the prepared sample solution is mixed with the assay reagent to form a reaction mixture;
g. heating the reaction mixture to promote a nucleic acid amplification reaction using the nucleic acid present in the biological sample and the assay reagents, the reaction generating an amplified nucleic acid and a plurality of protons;
h. reacting the protons with the optical property modifying reagent, wherein the reacting is capable of modifying an optical property of the optical property modifying reagent to allow detection of the modified optical property, which is indicative of a presence of a suspected analyte in the biological sample; and
i. causing a plurality of light emitting elements to emit light in a repeating pattern at a repetition frequency, so as to determine one or more characteristics of the biological sample using a photosensor based on the modified optical property.

83. The method according to claim 82, further comprising displaying the determined characteristics using an electronic result display mechanism.

84. (canceled)

85. The method according to claim 82, wherein combining the biological sample and the preparation solution is within a sample preparation device, and wherein dispensing the sample solution into the incubation chamber comprises coupling the sample preparation device with a sample receiving module, so as to create a fluidic pathway between the sample preparation device and the incubation chamber.

86-87. (canceled)

88. The method according to claim 85, further comprising breaking and/or rupturing a breakable seal on the sample preparation device, so as to enable the sample solution to flow from the sample preparation device to the incubation chamber.

89. The method according to claim 82, further comprising maintaining the mixing heater in a deactivated state until the sample solution is detected within the incubation chamber and/or until a minimum liquid level of the sample solution within the incubation chamber is detected, wherein the sample solution is detected within the incubation chamber and/or until a minimum liquid level of the sample solution in the incubation chamber is detected using a sensor.

90-92. (canceled)

93. The method according to claim 82, further comprising regulating the mixing heater based on (i) a constant or substantially constant power supplied to the mixing heater, via a power supply, or (ii) maintaining a constant or substantially constant temperature of the mixing heater or a portion of the incubation chamber.

94. (canceled)

95. The method according to claim 82, wherein heating the thermally sealed valve results in softening, melting, and/or dissolving the thermally sealed valve, wherein said dissolving is within the prepared sample solution, the method further comprising:
sequestering an initial amount of volume of the prepared sample solution and the dissolved thermally sealed valve in a sequestration chamber located upstream of the one or more reaction chambers.

96-106. (canceled)
